SEQUENCE LISTING (116 sequences)

SEQ ID NO: 1 - DNA, 3562 bp, Homo sapiens
actcgccggcactcgcccggctcgcccgctttcgcacccagttcacgcgccacagctatgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatagccaaaaattctccttgcctttaatgtgtgaaactgcattttttcaagtgagattcaaatctgccttttaggacctggctttgtgacagcaaaaaccatctttacaggctcctagaagctgaaggttagagcattataaaatgaagagacagacatgattactcagggtcagcaacctagtgagttagaaaaaaaattaacatagggccctataaggagaaagccaactatgttaagtttacgtgtccaaattttaatgaaattttactaacaattttaaaccatatttttcttcttcatatccatttctaatccatcaaacagcttatgtttacataaaattttatcattcacaaggaagttttaagcacactgtctcatttgatatccacaacttatttttggtaggaaagagagatgtttttcccacctgtcagatgaaaaaactgaagctcaaaaagggttgacttgaccatacagctaatgctgacagatccaagacctagacctaggtcttttgaactcaagtccagcattctcaactatatcaagttactgttcagaatacttaatatctcctctcttcataattatcaatagccccaagctcatggatgacaaatctctgctttatttcttgtctctattttttcactttatagctcctgttataatagcaagtttaatggtataaacacaggataccatcctctcttgcaacacccatgtgcctttgatgagtcaggtagcaagctgtagtagataatgagaaaggccagaggctgcaaaagacagtcaaaggacacgagagaaaggaaggggaagaacaggactccaggactgttttatattatagaaaagcaagagctaaagagcatttacacatgttaaacagatacttgttaagcatagtgcctgacacacggcattagctgttattttatgagattccatcagctctgcctctgtcctctttcttctaacatgaaggtatcatgagaagagaaccttctaacataagctgtaattctaaacctgcacttgtccctctccagcaagaggctagcactgaattcattctactcatactacacacccagttatggaatgtccagagttctcgaagaaaataaatgactttaggaagaggtatacattttttaagtcgctctgcctccaaatctgaacagtcactgtaaatcattcttaagcccagatatgagaacttctgctggaaagtgggaccctctgagtgggtggtcagaaaatacccatgctgatgaaatgacctatgcccaaagaacaaatacttaacgtgggagtggaaccacatgagcctgctcagctctgcataagtaattcaagaaatgggaggcttcaccttaaaaacagtgtgcaaatggcagctagaggttttgataggaagtatgtttgtttcttagtgtttacaaatattaagtactcttgatacaaaatatacttttaaacttcataacctttttataaaagttgttgcagcaaaataatagcctcggttctatgcatatatggattagctataaaaaatgtcaataagattgtacaaggaaaattagagaaagtcacatttagggtttattttttacacttggccagtaaaatagggtaaatcctattagaattttttaaagaactttttttaagtttcctaaatctgtgtgtgtattgtgaagtggtataagaaatgactttgaaccactttgcaattgtagattcccaacaataaaattgaagataagctc

SEQ ID NO: 2 - DNA, 45702 bp, Homo sapiens
actcgccggcactcgcccggctcgcccgctttcgcacccagttcacgcgccacagctatgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggtaagacccgagcccgcgcccgggatagtagtcccggactgagagaggagccgggctggaaaagcagcggatggcagagtgtggcaagcctaggtccagggcgcggagagatgtggggataaagtgagactccggccagtgtgccagctggatgcatagaagtcccttggacgattcgtcttaaacggacgttattgcgcccccactatgagatgggagtatttagaggagcaggacccagtcctgcctgcgaggggcttaaggtggggtgcgtagaagcactgacagttatccagagctagggactgaatcggatccctcacgcagctctcatttactgctagatctttcaaagaatgctttatctcaatctagatctttcgaaaaatgctttatctcaatcttattgaaagggaaaccgcgtcgaagaagctgaataaattaacaggcaccatccccgcaaaaagggagacgtgataagaatggacaccagatctctttgaccccggagcttgagagtaattaagctcacagctttggggtagattctctgttcaattaccttctatgtgaccttgagcaagttatttactctttcttgcctcagttctatcctgtataaaaataaggattataattgtacatagtgcacatagtgtggtctctatattattaccagacacatagtgtggtgtctatatatgttgttattaccagacactataatatagggttaagaaaaggaaagaaagaaagaaagagaaagaaagaaagaaacttcagagagagccaaaatgtggaaagacttgggtttcaaaggatttcaataaagtaggtttggcggcagagatttaggaagagtccaacttaagagataatgggctcttaaaacatgtgcaaaagtttagttcaaagtcccaaatgttaggttaaaggtaatgccttttctattgcactaaattctagaaagggagcagaggtggttgcacagtaataaataagtacaaccaagatgtatatatggtggttaaaaaaaaaataaaaccaccacccaaatttaaccctgtgtaaaagccttgagtaaagggattcagcaagactgtggagaaacttgggagaagaaaaaagagctttggaagaggaagaactgagaaatctccagagaaacgtccagagaaatctgtgaagtccacaagtgtggaagggaaagggaagtgtgattaggctgggatacagaaggctaacagtacactttatatgagaagagcttagaggtcaggagagcacaatgctgaggggtttgtttggttttattttcccctctggaaacttggagaaaggaggacaggaaaagatgataagagggcaccattgtttggttttaaaggtccctccccaaccccccacaacaagaaagctcattttaatgagatgagagaaggggagaaagctccctccgttctgatgcttttgagcttatcagggacaccctgcagcagaaattccagctgatccttcagccttcagagttgatgaggtggggctcacacacatctggatttgaagaagaatcgtgggtttcaagaactctgtggtctcttaagcattggtattccacagctacacttccctgatactttgcactcagtaatgacaaaataaatatttgtagaattgagttgttgctaccctgtgtctccacttcctcccagaagggtagaataaacttacatgtgtttgttttattttgcatataaaccacgactttgtaaactttactttttgtgacactcttttctccacaattgtgtgaaccctttttagtggaaaaataagaatgaagaattcctagttttccctaataatatttccctcttgtaagtctagttgagctcctggtgttaatcatggtatcaaatgtttatgacaggacatctaggtgaggcctgagtaaactgctgtaagttacagcctatctcttctgtgtttgttcagaatcctttttgttggatgaaattactttctttcaaattttgctcaagaaaattgaattgggttctgaggaaaaataggctacttacgtaatgttggcagggggtaaagattgggagcggatcacatggcaaggttagatggctgtgtcctcaggacttactctgtgcagtttgggggtgtgtcaaacatgatttgacgtgcataaaatcccagttctgtagtgctaaagcatgtgctaaagctctgctgtactccagtattgcacagatgtttcactgtcaattgtttatattatactaaccctcagagaatttattgctgttgtggtataaacgttcctccactgtaaccactcaccgcatattgattcctaggacactggggattcaaatcagggtggaaaacagttgtggaggggcatctccagtttcctggagactggacctagttcaaaggagccacataaaaggactgaagcatatggaggacaaagaggaatttcctagaatccagctaaaacacaggagtcccctcgagcaggaacttccaaggaggtgtacactttctagacctactggagtaatcctgattgctgtagaggacccagggcaatcctggagaccccctggaaccactactaggcagaaaactgggaagaggatggcaaaactctccagttagtgtagctgtctcagtggaaggactgttgtcccctgaggcacgaggaagacagcatggaagcactgacagtatgcgcttggcctggttactttctctgagcttcatgttccttagctgtaaaacaggagtgataacccccatcttgcagaggttttgtgaggaatacatgagataatccctgctggtgctcactgaaagtgagtttcatgtgccctgggtggggaggaggggaattgtgaggaagggaaggtttgggttgtgcatcagattatctgattctcacagcctggcacagtctaccacctgcctgccctctaccatcttacctaagtaccattgcctaaactatttttgctatagttactcattgagcagacttatgaaatctccccacagcacaaaactgaaggtatccacctccagaggtgagattctcaccaagacaggctgtggggaaggtagctttgagcaggtctctagggtcccctagaaaggtgccttttctctgcttggctgctgcttctgaggccctggctgctgagcccacttcctgcccataaggctgcagccttagccctagccaattgctcctaaatgggggaggcagcagccagggtgggaaccatggcccccgcctttaactcacccaggggacaggttagtcagatagatggtcctttgttcgccaccctccacctcccacatagacaacattctaatgtgtttgatggactgcatatagttttgtttctacatgcactttcaaaacctttgtgtgcttatattttataatggcatacccagtcctgttatttagccctttctgtttcttacttttgtcctgaacttattgtttttatgatttgtccatgttgccctagatgcccttatacctatacatcactgtgaattgtgctctgtgtattttacccatctccacgtattttacccatctgctccctggctgatgggtttggagatggcgtctaacaccctgcctccacaaataatgctgctccagatatcttcctggatggtctcaccagatgatttctacccaggagcagaattgctggccacagggcatactttgagtactacaagactggtttcctcaatgactgacatcctccattctcttccacctactcccatccccaaacaggacatgtaaatttctatattttacctatatatcatggcagtatccagctttattgtaggtgtaaaatacacattgctgctttatcatgtacttttctaataactagtgagtttggacattttttgatatgtttgttaactttttaagcttcctcttctctaattgcttattcatattctttggtattttttaaattgggttagctttatattttttgactttcaggagttataggttttgatctgtgatcaattttagacattgcaaatatattatgatgatattaggtctattaattttgtccatggtatctttcattgaacagaaattcttcatttcttgtaattgaattaatctttttttccttgtggcttgtgcttttgaaagtttgcctaatagtttgctaatttaaagcttatcacctttaacactcctcactggaaattcagagatcaccacggttctctctgccttttaagagataaatctacaatctgtggctatgcaattcacttctcagcatcaccagttcctcatctataaaatagaagtttgaaccagcttacctctgaggtcccatacatatttgaacaactggagccctacagtgaaatgttttgattcagtgaagggacaggaggatggggcaaaggtgtagaggcaggaatctacacaatggaccaggttggaccagagtgaggtagcaggaatcatggcagaagaggttgtgtaaactctaaaggccacctgagaagtttgagttatgttgccatgggcagccatgtggtttctgcgtagaagaccactgtggtataataaaaaataaatatttggtccttgtccccagtacctgtcacagagttcctaaaaccctagaaatttcctaagtaatagaaatgtcctttgtgattcataataaaccccatgataatacctgggtttatgctcctgcagacttagggtgagatccctagatagcttcaagatggaaccagtcaccagcaagtccaagagattaggggatcaaagagttggaactttcggtaccacccaccaaagtccaggaaaggagggtgggactggagattcagctctacaaaaactcttgaacaagatgtgaggagctttttagttgctgagcatttggaggttcctggagggtgcacacccaggtagggcatgaaagctccgcattgcccccatatcttggcctatccattcttcacttggctgttcatctatatcctttgtatagcctttataataaaccaatgagtgagttctgtaagctgtcctagcaagttaattgaagccaaggaggaagtaatggaaacccctgatttatagccattcagccagaagcacaacctcaggcttgtgatcagcctgaagtcagggcagtcttgtaggattgagcccttaacctatgggatctgacaatgtcttgagatagtatcagaattgaattcaattataggacactcagtttgtgttttctggagtactgtttggtgtctgggggaaatcccacacacatctggtcacagaagtattctgtgttcagtgtgacagtagaaaaagcagtttgtgtgtatgtgtgtgtttgtgcttttgtatgctaatgagatgatctatggctgggggcccttagatagcttcgggaagggggctggtcgccagaaagactaatttatgactagagggttagaactttcagccctacctctcccacttctagagggtagatgggctacagattgagttcggtctccaacagccagtgaattaatcaatcatgccttcacgataaaccttcatgaagacccctaaacaaccagattcagagagcttccaagttggtgaacatattgaggtgctgggaaggtggtgcacatggagtgggcatggaagctttgcccatccactcccatgccttgccctgtgcatctcttcacttgcttgttccctagtcgtatgctttataataagccagtaaagtgctttccaagttctgtgactcgttctagccaattatcaatcccaaggatagggtcatgggaacctccaatttataactagttggttgggaatacaggtggcccaggactctcgactggtgtctgaagtgggggcagtcttgtgcatttgagccttctcacttgtgggatctgatgctaagtccaggtagagaatgtcagaatgcatctgaattgttggacaccaagttggtgtccacacagaattggggaattggttgatgtgaggaaaaaccctcccagctacatagggaaatcacggttttggaagatgagcagaaaagggtgggagggagaagacaagagagaagagggtgaggaaagaggccaggcagcagtccaagtgtgagatgataacacctgaactctggtaattgaggagaccaaatccacgggaaccagctgcaaaaggcattctgtctggtaggtatagggctaagggagtaaacgtgggggtgagactgggatcaagtccccttcctaccacttagaatctgtggactgggagcaggttatttaatctcatgaaacctccatttccacatgtgtgaggtatgtgattgttgtctccacccctagggtgctgtggggtttacaagagatagtagtgcacataatgtgcttagcacagggccagcccctagtaagtgccggctgtacttgccttctctttgcaggggtggctcaggagagggtgtaatcaaaagatgattatgagggttcaagcaccaggcaatgggagagtcagcaggttttaatgcttccacacagaggctggattcttctacttccgcttaccgctgacagctgaggcctgctacctcaagccctgaaatgcagtctgaagacttttcctggacttactgggcaccattggactgtacagggctgtggcagacaggagcagtggaaagagatttggatgggacgtcagaacacccagattttacttcttggctcttccacttactagctgtgtgaatttggcagtggtttccttatctataaaatgggggaattggagagggtggtctactgcatgttacttctcagcacctcagctcctccccacccctctctcttgcttctaatctgcctacatcacttggtttgtctctggacaagtctaagtttgcatgacaactctctaatttctccttcacagttatttatttattttttggcagctatttccttgtggagagatagcgtaggtctttcaggtgcttccatttggccactgtttgcaggtgaagaggtttccaggatgctcgcgggatgttactctggtagtaacagaaatgatggcctggggaatgggaagagtatgagcccagttttgagcctgagtctttggggctcatttattcattcaatctgcaaatcttcattcagcattactacatgccaggctctctgctaagtgctgaggatagcgatcaggacctgctagggtgaaaattactcttcagaagatagcagagtggataatggccattcgagttcctcttaactacacagagggactgggtaagatctataaaggagagaggttaaacagcaaagcaaaagtgactgaaattctaacacccatgcatggggattcttgagctaaagatctggtctgtagaagaagacacatcagaccctcatatttagcatctaagccgaaatcagcagtaagcatgtttcttctcccataaaaacttgtccctaagaagagagacaagtgcttacaaggactcttatgttaaaggaaactgtcacaattactttatcttcagtcatgaagcctttcttgaggaaagaaaaattagcaatactagtttgggaagcttgaaagagagattctgtttgcaatgacacttttaaaaaggcaacaacaaccaaaaaagtaattattcctaaagctcaggtgtcagaagttgtgaatgtctagggggttgtggatccccaacctcagagacaggaacagcaccactcccatcctggggctgtggcctcaggcctctgcttcctctttttcagtctaaattccagagcagagaggtgctggatgaccagcttcaggcaggggagggaggaagtaaaggtagtttctcagctcatcttgtcctcgtactattaatagcatctgttgttttcttaactgaaaattggagagaaccaaacaaatcaacatcatattggcggctttctgcacatcactcacaaatacatctgaatgctctgtactctccagtgtttattttcctcagccacataatcggaatttgaacattcttttgacacttcttttccctcacttgaataattagttctatatataatccaaatattagctggagccctctagcccctcagagatatagttgaaccttcctggacctcaattagttgggcagttaattctgaatgttgatgtgcctattttgcacgtatggagagtttgtacgtcccccctccccaaacaagtagaatgctgtctatttggtacttgctcattcatacattctttcaacaaccaaacccagttgcagctgggttgaaccccagggtgtttcctgctattcagtgtgatgccacattcacctgctgtggccaacattccttggcccaggactgcagggatggaagccaacccaacatcagaagtgggcaacaatgccagaaacctatcaaccaaaacaaatgtaatttgtaatcctccctggatgatgttgagtgattggcaactgagccccgtcatatagtaaatcaggctgtctctctttaaaaaagagagacagagagagagagagtctcccaatgttgcctaggctggtctcaaactcctgggctcaagcaatcatcccttcttgcctcccaaaatgctgggattacaggcatgagccaccatgcccagcctaggctgtctctttagctcttcaattatagggcttcctgcaacctcagcccttaacgaatgagctagcgtgtaagtgactagctaaataaatgtgctgcaaacctctgacaaatggctttttgacattattaaaatttatttatgtggaacaaaaaaattcttttcttctgcatcatgcatcctacactgcttttaaagaaactctgctatcctttgttttaccgtctaacatttctttgtttcccttctgacatgtttcagcctcttgtcatcacttggaacatttttactgcctcatccctctagtttccatttcctagcctagcccagcccagtatttttgaaacccaaatacctggaattttgcctgatgtttaaagccaaaaccaaatgaaaatgtgaagaaccaaaggagattttattcttctgtgactcaaaatcctgttgttattgttaaatgaaccctgtgggatacagtctggtatttttaacaccttctgggggtatttgtcaatatttacatttgatgtggatagcttataaaagtccagtggctttgggtaaaccctgttcactaagaataagacccagaaggtcatatgaatcaacgatcaaagaatgaagatattctgaatccaagtttcttgttttgattattaaaaataccttgcaattagttaggcatgagagaaagcttgtgtataaagtttggcttgtaattcacatgaattctctatacaggccaaaaactaccttccctttgggaatatacaatgaccttatttggaaattacttcaggtaattatgaagtaataatatctctattcatatgctaaaaagaattcaaaataacattcaattatcatctcttagaaactaaattaaacgggtactcacccaagttgaaatgcaaacaaatgcatgatatcattatgtgaccctcccctagcccttaccctgtccatggcagatgctaagaaaaacttgtccctaagaagagagaagtcaattgatttagtcagcctttcccactgtgtttggactgagcctcagaaccctccttatccccatactgcaaggagttactgtcaatcaactagagatggcgctcaagataaaattattttccatcctgtactaatataataataagcatggaatctatttattggttttttcacaacaataaaatatatctttagcaagtaacaaatggtagaggtaatacttttagataaagagggaagaatccttctgacttttctctttatcacattttacccacacacaatgtggatggaatctgcaactgaatttagcagtcacactgaaactctgctgcatgaccacactgtcctgaggccatgtgccaacttctatgtttttttttcccaaagctcttccaaggaagagctgccacttctccatttactcttttcacctgtaaatgaatgctgccgttggtattgagaaaatgggtgaatgaaagaaatcatgagaacactcctggggtgagagcaagaggaatgaaaacacgtggtgtgagagcaggtcagagatggtggttaacacaggaaccaggatggaggcctcagacttgaccctggcctgcaggacgttggcctgcattgtctgcaggaggaaacacagaggtcagtggatatcctgcggtcttccattaaggcctcttactattctggaacattttatttttatagagaaatctcccacttcctgatctccccagttagaattgattgatcaaagcagtccctctccctgttattaaataattaaataatatgcattagttgatccctataaccacttaaaatgtatctaacacaggagaaatctttggtaaatatttactgaatgaatgaacaaataaataattggagggagggagaaagagacagagttaaatcctgaggctggagtttttggtccttcagccccttatcccattgcccctggcagccccatccatgtgaccttgaattctgtccaagtctcatcactttctatgtgtgttgtaatggaaaaaaggttacaaaacacagtctttctggcctggagtaccaaaatagtaaagacttctgttttgaaagcctctgacaaatgaacacaacagccacagtgataactttagcaggaaattgaaccacaaaaagcaagcgagccacaaagccctgaaggctaaaaatgcctcacaggccaagaggcagttaggttgcacctgagacagaggctgctttcttaactgggaaatgggtgtctccagatatataaagctgtgaggagatcaccatggcccagctctgacgagccactggtacagcattgttggttagtttgcctcaacctgcagcagagtttttattccaagagttaacagttagttaggctttttccaaaggttgagatgacaccccaactcccacacccatttccgtcctgccccagcttgaaaccaggcaaagtgagttaagacattgggagatgggaggggacaggaggggtagaggcctggcacccctctctcttctgtttcaagagacactttgtggagttgtctagagagactgggggtaactgctccttctgcccccaatctccccttagctccccactcctccctatcctgccaggccaaccaccagcagagaagggcgcttagctccagtgacgctgtgagtgaggtacttgtgaggatgcatatggttcctgttggaaatgcacggaggagcagtgaaacaccagaagccccatcctctctcctgcccgtgccctcatgcccagccagctgaatattcctgtggcttccactcctgcactgtcccctcagtgtcacctccccaccacattcccacagccccacttgctgtgtcacctctcccctgagggagaggtgtcttcctgccagtctcttcaccaccaggctcacacaactcccatccattcactgcaggctgccagactgagctttccaagcacccccggactgcctccagccctactttctcttcaaggcttcccacttccctcaaaatgaaagccctgactccttggacaccatacagaccctccctgatctcccagcgatgctcttagcagcatcatttttttttctgccagattttcaggtgccctcagttccaggagcaccaaatcacctggatgccatgagctgcttatgacacagttcaaggtgtgacatctttgaggtctctccataacacctctactgcagacgtctgtctctggtgtatcagctgtaccttgcacacatgtctctaaccatctgtccactgtgtctgtctgttcacacatcagattgaccctatgttctctgtgacaccaggtgagctctggcaccagtgcccagcagatgattactgaatgattaataaaactactcactccacctgctgcttttaaatgggctccattttggtatgatttggtgcgtgaaccattaaaacttttcagaagtcaggatttgaaacaaacttggtaaaaagtagttaagcaatgtcttcgttgtaaagtgagaaaccaagttgcttggaaacaggtgaaaagcttagccagcagcttcacaattcacagcctggcactccagtagccctctctggacaattgcccaggttttcttgttttcatttctgtgttttaatttacaagttacctttatatttctactaataccttactggcacttgtttgtgaatgaaacaatttttttgagagtttgttaattgttctgtgtgagtgtggaaagtttctttcatataagatcatcaattctttttttaaaaatagtgttttaggttttctcctacataaagaaagtggtgggggtgaggagctgggggtggaccagatgctcacattgtatttctatgattttgctgttggagatactgaagatcattttgctgggctttgctctcttaaattacagaaaagtgcatttggtgaagtttagtgtttttagtaaacatatacaagatgtcatagggatagaattagtgaaggaatagggttcgtgaatgaatgagctcaatcttagtaatgaaatgtttggaaagcaacagaacaagagagaaagagcatcagaacatgatttaggtaccccatacatttatacaaataaaaaaaagatctgatttagggtcatagaaatcacatacctaaactgatggttaaacaataagtggcaagtaacagtttagctacaaaaaatgtacaggaacacataaggaaagttacatgggaactttcagagaggaccaaaaaaggtggaataagattatctgctctgttatgatcccctgtatttctctgctctatgttcttctcttgggcagtcttttcctgagatgttttcatgttattaatcaaaaataatagaaacgaataaactagatcaatgcatttacaagattttttgagtgcctgcagtatgcctcggtgtggtgggaacttggtgtactgaggtgagtagcatgatcctgaagagcttaggaatcactgagttctcgtaagtgcaatagaagaaaaactggtagaaggaagtcaaatgaaaatacaagtttctaaagaaaataagtatttacaaaggaatttgtctgcattaatttctgtggaaaagttagctatgtatagaagaaagaaacttgttagtaattctttcattcattcatttattcattcaacagtgaatccctgccatggtagtactaactacttgcactgtggtagtgcaagggatacatttgctgtgcaagtagttaggaattttttttttttttttttttttttttagtgctttgtgtatgctaggctttatgcaaatatacagccaagcacagaggttaagaactttggaatcaaaccgaagtggttctagtcccagctcccccttacaaattgtgtgatcttcaacagttcatttaacttctctagatctcagattttctcatcagtaaaatgagtgcagtaagagtaccaccctcacagcatgaaatcagattttctgtactctttactaactcactttccatgacgctggcatgtggcaagtgctgactcaatactggctcatttccttaactgcttttattttattcattcactgccattccagaggaaactgcggttctgagatgtgccctgggtcactgctgaggagcggacggagcctgggagggagctacccctgccttgtttcctcacactttatggggtgggtgtcaggaatgaggctggaggggaaggcaaaaccagatccctaagagccccctggaccctgcttagggtgtttacttctatcataaaggctgtgcttctgtagagacctaaggcagagagtgccaaggtcaggtttgcatttgagaaaagtttttctggcagcaatataaagaatagtttagcaaaagaaatgagaatagctggaaacccagttaggaggctgtggtagtccaggagcacactaaggcagcaacaataggtggatggaaactggagcatttaggtaagggagagggatgccagaggacgtgatggtgagggagagggaagagggtgggactccaccgcaccagggcttctggctttattgactgggtatatggtggcatcctgggccaagacagtgagtcacagggaagaggctttgagcctggtaagcttgggacacttttgagacattgggaggaaatgacacatggcagttgacatacagatctagacgtcaggagaaaagtcagggctggagattttcggtttacagttgaaggtatgagtaggaatgagatcatttactcagtccatcccaccacattgagcaccatgaataaggggctcctctccccatggggcaagagaggtgaaaagacagtcaaagtcactactttcaggaggattaggctcaagttaggatggggaggtggagaaggcatcaaacaaataaatgaaaactataacttcagttattgccaagtgctaggaatataatgttgctgttaaactgcaaggtggctaacaagttggatagtctttcttgaagaactgtagaaacattcattaaaaacaaaataattttatagttaagcaagaaagtgattgtactataacaagggagacttatttcaccataggaaaaaaatcataacattctaagaatagaaggaaaaaataacaattacccaagcctgcaacaacctgtaagaccagatcagcttttaagaggaaagttaattagataacaggaagaggtaaaaatgagatcatcagagatgggggaaaaggcaaatttagaaattctgaatttaataggggagacaagtatgtaggaatataaacacattgtgctttctctgcacaaacacattcacatgaggatgtatacacaacccttgcaggcacaggtactcagtgaatgcctgcgtgccaaggagggggacagcactgagcaggagaggtggcagggcctggtggcccaggagcactcatatagtcaggagactgatataaaagaaagatcctgctaacatgccattacagagtatggaatgagcaaggaaggaagagggtgaggctgtaagagagtgcacaggccctcatttaaatggacgggtcaccagccagtacttcatgcctgttcctagatgacccacaaagtgaaacgaaactggatgtggtatttagcaagtctgagatttgctggtttaagtactaagttagcaaaccagccttggcagccagagcaggtagggtagggggcccaaggagatgggtgtatccaggaggccaatggaggaaggagaggaaggttttctgggaagccagctgacatgtcatgctttgtcagtggggcctctgcatggattccctccactcccccccagcccccaactcactacggggagctgcaaaatgacagacaatgggtggttttgtacccagcagccactcctcaaccagtgaggaatgggagagagataagaaggtgggaaacaaggattgcttgtcaccatccctgctcctcctgaagccctgcgctgggagcaggtacagaacccgctgaaggtcacctaatcatttcaccatggagttcttccccagatgtgaaactgctgggactggagcaaagggtgcccagagctgactggaggaggaagccagagccacactggaacatatggagatgctagctcctgcctgctattgaggaacttcattttcccagaagaattagaggaccacaggagtgcttgggaagaagcaattgtggcaacctaggtgaatgatgacctaggaggtgaaatgaggaaggaggcctttggtctctgccaaccatacatttcaggttgccttttgctaaagtcctgctgatgttaatcattctctccagcagatcgcggcttgaatcatggggtctgaaatgtgggtttatgagagaaaaacaacatggaaaaattgggcagaggctggtaaatgtgaacattcaaacctgcccccaatctgagatggtgggacagtggacccctggccagctctccaggtgttgcgtatgcacattggcaaaccacctttattaggtggtgtgacacctgggtagcaattctcaagcaatgtgatgatctgaatgtccctgggcctgacccaggtgaggaggacacaaaatcatgcaatatgtctcatagagcttagtcagttttgagaaatactggactaatgttatgtttttaaagcacctaattctttttctcttttctgttttatctaggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccaggtattttctacttttatagcactcaatgcttgaaaatagatgccctaaatcacagcttggcattatatttatggactgtagataaaagtaggaacaactgggtaaatagtgcactagaataacactgataaaagcaatctggtaaattctatgctgttgttccaaggtacttcttaaagagatgtcctcttctgctcagtgaacttctttgttgacaaaaacagtatctttgtctaattcagtgctttcaaaatgggaagaggagacaatgtatagccagtgctaaacagtacagtttcatttactagagaatgagagcctcaaaactcactgcccttctcctgtggagccatgagcacagagccagagttttacctcaatttaccttattgctccttttctaggatgattattacatgtccagctaactcacctttctcgcttttcattattcatctcgttttaatgacattcttgtatctgactttctgggaaatatatatagtggtcttatgatatatatggatatatataagtgtatgtatatacatttatagacatatatacacatatgtatatataatatatataatcttaagacacacacatacatatatgacacatatacatacatacatataagacacacacacatatataatcttaagacccttcaagaaactattttgaggcacttttataagaaaactttttggaatattttggcaatgagaacaaaaatgacattttctttaaattatccaaattaaatgtgtttccatatttgaagaaaaaaagggaaaataggagccagggttatctagaaacatgagttttcattaagaactaactgataaaggacatataattgacaattcaaaaatatatatttactgttgttgttgttataaaataaataagtccaggccattggaaacaacaacagaaaagtcagctgtgtttgctgagagcagcaagcgggtcctgcagctggcatagaggcagagctggaggcagggtggagcggctggggctggagtggtcagtgagaagagtgagagaacttgctaaagaaccccccaaccccaccctggggtcaggtttacttgctgaagaacctccttggtccccttgagagctgtgtaaaacatccctggggcacaggacattccagaagtgaggtcagagtcacaagctaggatgcaggctccatgaggacagaaacctctgctgtctcactattgcccaatgactgtgcacagtgcctggcatatgataagtcctcaggaagcattgctgggatgaatacatgagagcaggaaggtataggggccacagtcacagaacagcagatagttccatttcctggagcatgggttctgactaggaaaagctagaaagttggatcagggctagattgtagggctgagaagttcaggtgtgatttggaggaaagcattgtctcttctttaaaaggcacccataaggaggtaggaggtgcaggtcttggtggaaaggcacagcagctccagaaatcaaactgccccagctgggactctgaccagccctttaccaggctgtaacagagaggggatcaaaagtgacactgctgtttccagcagctgctggaagagagggctgcattttgcagggcccagtgtggggggaaagggatggggtggaatagaatgtttccaggagcactgcttcttccaagactacaggcccagcggcctggtgtgggttcttcctacagttctttgaccctaatcaagcccagttctcccagtcctagtggcctgggcacccacagggttataacctgtatttctagcacatttaatctaatttattttagctagatcattaaatacctgaaatacaactctcctcatgacatcttgaaatataagccatgctttgtatatatgggatgcctggtcctgtgaggtttctgatataatagaggaatacattcctttgtcctcagaggcatttctctgaattttttttccttcttggcagccataccttaagcaaaagatggtagcatactctcgacatttagtcattcccctgggtacctgtctgtgtgccaggcactgttttaggcaccagggatactgaccaaaaaaaaaaaagttgcccactggagcttacatattcagtgggggaatcaataaatgagtaaatggtttggtatgtcagaaggtgctaaggctgtggagaagaattaagcaggaggggaatggcaagtgcagcaggggggaaggtataccttgttgtttcaaagagatgctcaaggaaaggctcacatagaaggcaacatagcactgagggtcaacggggagccctgggcttcttggggcaaccaacaggaagcagattgtccttgatgttctcataatagacaacagtggcaaaactacaactcttgatttttcaacattgcctatgagttacagacagccttgcctgtccaacacatctgtgagctgctaattattaggttgtcattctggggcctccctggctatgatgtcagccatgaattatcttccatggtaatttgcacttgtggagttggctaatgttaaggattcaggagttcccctgctgaagcacacagacattcctaaacctcacagtggaacaagggatgcatttctaataatcttgttactctttggattagtacttttcaaactggagtttgcagactcctgcattttgaaagcttttaatgaaatgtcattctcccaccctccttcccttgtttagtgttagttcttagaaatcagtgtaagaacgtctggatctactggaggactccctagtcacagagtaccagacatgacttcagggcagcatgtctgtatttgcagtcatcttcatgccagtgatagccataaaacctcaaagcttcctcaaatgtggctaacagagccagttggaggaaccagttgccttgctgcacacagagaacctgggttgcacagtgagagccatgcttgactactgataacaaaaggctatcgtttgttaccactatgtgcagtaataagcacccagggaagaatgtacacaatcccagtgccagcaacatttagatgaggccctcatgattaacacagagctcaataatgctctaatgctggcattatttatatatttatatattttttgaggcagagtcttgctcttgttgcccaggctggagtgcagtggtatggtctcggctcattgcagcctccgcctggcaggttcaagacattctcctgcctcagccttccaagtacctggcatgtgcctccacgcctgaataatttttgtatttttagtagagacagggtttcgccattttgcctgggctggtctcaaactcctggcatcaagtgatctgcctgctgcagcctcccaaagtactgagattacaagcgtgagccactgtgcctggcctgctggcattaaagagaacatgacaaagttatcaactgccatattaaaataggcattcaaggactacttgctgacagttttaagtttgaacactgattatcccaagtttccaacgaacgtcttggagttgctaatactcttttgtgtatcatgcttacgtgcttaggcattttctgcattggtattgtggaagttgagatgtacaaatatataaaacaatatctgaagcctcctcacagtttgcaaccaagttcaagcaaaacaatatccacaaccttaaactaatacaacataaagtgaatgtaatgaggatattgatttgaatttttatttacttcttaggggttttgtatttaatgaataaatatgttttggttttactgactcttgagctttagtaaattaaggtgtttaacctttgcatgttaatatgtatattaagtaatataataaaaattgttaataaatatccattttatttattttgttccttagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggtaattgtttcaaaaggattgcatgggccaggatgtccagataagcactgtgtctcttttgcctttgtaactgttattactctttttactgctatttaatatgtaatgtatattatatgatctataatatatatgtaatatacattaaatgggaacatgtgcaaatcttacaaacattactaagaatttattgatttcatgattataagtcaattccatatatttatgtatataataaatatataagacataaaatatgtataaaacatatatataccatacatatgaaaaacaaaaagactattgagccccaaccaagtcatttatgatagattcttatcaaaaagcaacatgtcatataaaaactttaagcataaacaaaataatgtaaataaggacctttataactcaaggtggatttgatttgtaggttgcacaggtagcagaggctggacctcgagtgtgagtcctaactctgctgcagactaggaatataagtatcctggaagccacatgaccctgggtcctcaggagagcatgcactcttcctacccactacctgactacccccaccactctctttttttctctctcaaattggtgttaagtagttttcatcagaataactcttttatgatttttccactttaaacctataatgtcccaaaacttttgaatctggttccacctttcccacttcccccaccttgcactgcatagatctttgaagtattcccaaggaaaggaagaagaggctggtaaacagttcttctatcagcattcgcaaagaaggctttggcgtgggaacagccctccccttgaagaggctcctgcttctaccatccttatgtgggctgtcaaagaccatgctattttgataaaagcgaaactcaaaataccccaaacaataagtaggaaatacccaaaaacattaaaagggattatcactaagaaagtaatttttattttctttctagtactttggagtattttcaaataatgaatgcatgttacttttgcaatcggaaagcaaaatataaattataaaaaggaaagaaacacaatattgggtcaaaagtccccattcctaattcacttgaatgactttgagcaggtcacttagcacctctgaatctgctagcttagcttcaaaataggggatgatcatgctcaaagttccacgttactgtgaaaattatatgagatgtaaaaatatgtgaaagcagtttataaactataaatagctatattctggtatgcagcgtcatggaggttgcctgcttataaaaatatgtcagtcaaaatcataactcacaaactaaagttggccttcacttaagaaaataccagtatgaatgtgtgctaattaaattccttcacagatattttacttcttggtaaacacaggctaaggaataatttcaaactgagttaaactgacaggttgtgaaatcaatttatgggttatgaccagtagtagggcttttttttttgacagataagactaaaatagattaggcacttacattactcataagaatgataaatattgttccatgggaaaagatgtatataagatgtcaagaacagccccaggcacatagcagacaatccataaatgtgatgaggatgacgactagcactggacattgttagcatggataacattgcaatcaaagagtttcaacaactccatgagggttaaatgaactaagagagccaagacgaggaaataattggagcaggtcactgggaagcactgggtaggggtgaagactcaacaaagtccttctgactctaccactaatggtggggtcctttcaatgcctgattaccctcatctgtggaatgaaggtgataacaacagtacctattataaggggtcactgtgatttaatatgtgtaatgaatgtataggtaaatggccttagcacagggcctagtatgttagctgttcttattttagttatgtgctggctcactggataagacctgtgaaaacccaaaccttagcttttcactgaggcagtgtcagcacccttggacacacagtgggagctagatatgtctcctcccccttcttcctttagaataatggtttcaaaaagtttaaactatgacccgtagtaagaagaacattttagacagggagccaaaacacacaaacatatatgtgtgtgtgcgttcacatacgaacatgcaaaagcacttgtatgtgcaaacacatacaagtgaaagaaaactttcacaaagcacatggaatgcaccccaacttttctgttttactgttttattcacttaatgccagctaccactcattgaattgattttcttgtactgtagttaatccatgtttctaaaaactcagctttaaatgggacgtgaccaaaataatgcatgaacactcaaaggagtttcatcccttcattcagatgtgaacattcctgtgttccccttgacttgtctttacaccatcttaatcaatgctgtcttacagcaacaattagtcattcatagcctgtggtttgtgtccatcagtgagtgtgccctgatagacctgaaaagattgggagaccatgcccagcttcccattatttctgtagcctgtcctttgttcccataacagtacagtcatgccttagtatctgtgggggattggttctagaactccctgtggataccaaaatccatggatgctcaagtcccttatataaatggtgtagcatttgcatatgacctgtgcacatcctcgcatatattttaagtcatctctagattacttataatacttgatacaatgtaaatgatatgtaaatagttgttatactgtattgtttagggagcaaagacaagaaaaaatatatgttcagtacagataaaaccatccttttttagaaaataactttttgttcatggctggctgaatccaacaatgcaaaacccacagttagagacagccaactgtatgtgcctttgctaagtagataaatttagttaatgatgaaggactggatgaaagaaagatttgaggataaaaattagagtttgcttatctgtaaatcctgaagtaaataattacagtctctgagaaaactgagataactaatacttagtcatttacttcacatttcatgattgcttaccaagattaaacttgttctgatgaaaatcttcacttttcatactcatcgttttgatgagtgaacttaacaattaaaggtggctgggcacagcagctcatgcctataatcccagaattttgggaggctgaggcagaagaattgcttgagcccaggagttcgagaccggcctgggcaagatagggagacccccatatctacaaaaaaaatttttaaaaattagccaagtgtaggggcatatgcctgtagtcccacctactcaggaggctgaggtgggagaattgcttgaatcggggaggttgaggatgcagtgagctatgatcactccactgaactccagtctgagtgacagagtgaaaaccctgtctcaacaacagcaacaacagcaaaaacattaaaggtaattccaggacacaatagggatgaggcatattgcctgtttaaataaactttcctggccttaagaaaacaactctgctgaataattttatggaattagaatgacattaaaataatgaatctgataaatatgattagttaataaaaatacagttaaaataatacatcccttggaattttaaatcttactgagaaccatttctgcacgcacagtatccaatatacaaacacacatggaagaaaacctttccaaagctcgtggattgcaccctcatattttctgttctgttatttgagtttttaaatgctggttaccacccattatattaattttattgtattgtagcttgctcatatttttttaaaactcagcttcaaataaggcatgactaaaacagtgcatgaatggatcccttccaaatgttgaaagttattccagtttctttcctttccaagtaccatgcatatgcagctaaccctagcattggctgaccagtctaacatcagagtgaagagaagggaacttccctctacaaggcttacaaaatgataaagttacaaagataagagctaaaatgcaaacgctttctctaaagaagttaaatcctagagtcacgaactggtccatgttgcttcctttagagaaaaaagtagtaaaatcagtgcaggttgaaacctgcagcaggtacaacaggggactctattctctgtgctgtccttgaagggactccaaagatgctgtttctctgtttgcctcccagtattttaagtgagttttggattctgcaaatcaatctggctagctatggccttgggtgaatataagcaccagtttctatagccgcaagttaaagggatttgaattgttaattctgtcatccctcccattctaacatcctgttacctagaaaatcatccatacatagagcctgaaagcttacttttcaggatttggggaaataaaaaaaaatttggtagaatacctcatactttcattaggctactgatatgaacttaattattcctctaactcctttgaggctcagaccttctgtcccctgtcttatcactctcatctaatcacctccaagcaatgtcccacgtcactgagaatttagtatccaatttcctatcttctgctccactccagctcctgccttcacctggcagagtgtccatgtgggtagttgatccaataccccaagttcagagtcccttgatctcctcaatccagtgactgacacctccactccatttcagcaacttcacttctggggccacaggctgagacttgattatctccagaatttctccatgtccaaaactgaaatgggatagttcccttgaccccttcgtgggactcatgaaggggttgtcttgtttactcatcctgcagctctcaactcctcatgggagggagagcacataggtgagtgggtgcagagggtgggacaagtgcttctgggtgctggcaggagtagaactctgtgcggccccgcagcagcatctaggtgagtatctgtgacccctggagccgcagagggcatgtgttacagtgtgctcttttagctttgctgtccatggacagcttaagtgttttaacagctcagtgtgacagccctctgtatcctgaactcttgttcagcaaccaggaaggatcagcttgcacgaacaaattgaagatggtgaatgtggagaactttattgctgatgaaaatggctctcagtgggatggggagctggaaaggggatggagtgggaaggtgtctccccctggagttaggccatccccagctgaactcttctccgaggtcctaccgtcaagccatccctctgaagtcaagctgcttttcaccagtgtcaagcttcttctcttctctccttctctgccattccactgccagtggatcctggggtttttatggggacaggatggggggcagggcgggccagggtgattttggaaaaggcaacattcaggcagcaaaatggggatataaagttctcactttgggccacgggtccaggcttgagggtggagccattgccagggaccccacccttttctacctagtatttccctgcctccactctccaactctcttatcacaggcctcctatccataaatctttctcactcccttattttttttagagctcaccttcagacttatgaaagctcaagtcttttgaattcctctttccctcccagtccagcaggcttcacttctctccttgctagcctggaagtgtccattacacccttgactgtgtcgcggctgacacatggaaggcaggtgtctggtattgttaaatgaacgttaacaagtgcaacttctttaaaaagtaagatttctttatttgattgatcagttttttttttaattaagtatctaatttccattgcttctggatttaggcacacatattgaatataaaattcaaaacttttcatgtatctaaagaaataattttaatgaccacagaattttgctcttaaaacaaatggagcttgcaatataattataactaagacagactacaagcacctgctgtgttttcttcatgctgccatcaggctggtgcatagtctgatgggatggatgcatccctgctgtaataggaataagtaaagagggtgggagactgttgttggtgtttacaatcattattcatctctccattaattttcatccttgcagttgagataatcttgccttttagaacgagaaaacaatcttattacttacaacattcacaagacagggaaacactcctcaacaaagaaccatttcctttgggcatttgataagaactgctgacatctgtgtttattaagtgatgtgggagaatcagccaaggggcagaggtgtgggtggtagcttcaaaggtaatttcctgactgagtgtggttgggcatgttggcaatcccagtgtttgcaattccagtgctttgggaggctgaggcaggagcatcacttgagcccaggagtttgaggctgcagtgagctatgattacgccactgcactccagccagtgtgacagagagagaccttgtctcaaaaaaaaaaaaaaaaaggattttgctttcctggtctgtttgaatgtggtaagcacaagaacttatttgtactggtggtcacccacttgggtcctttcagtaaacttctggccatggcccagcctgagcacctgcttcctttacagagcaaagccagagccttctggtttgtaatttttgcagccacataacactgatccattttagctgtctcatgaaactgctttccaagatgaagtgagactatccgaaaccaaaatctaaaaagaatgactacagaacgaatggcagtccaaggtcatttggcagggtagaattataaggaaaggtgatggccagctactgtgaagtcttaagcccactcataggtacgaatctcttaccaggactgaagtttgtgtttccttaactttgctgcttgatggtcttctgcaaaggatctcatttctccagctttcactgggaagtggcatggggttttcgaaagtgctttaaactctaagaactggtttcctctcaggcttagccaccactagctgtctgactttggacaagttactcaaccactctgagcatcagctttctttttttaaaatggtgacagtgatatggctaccttagagagttgtggagattaagtgagataatatatgaacaaaatatataaatatgtctatattagacatatatgatctgttaaatatatttatatatatatatagaaagaatattagagaactgacatatttatcttagacctactatgttctagggcccagttctgctcatgtattatctctctatatatatagtattgagagagagaaagagagagagagctagagaaatagaaaccagtaggatatatcctatctgaagaactctaatacaggtatttcactgaacaaaatggcttctccttaaaatgtattccaacaccaatttcccctccttgcttctcatacatattaattcacagaaattatattggtcataaggcggtaggactatgaataatttttttcttctgcatttgccaaatattctgtaatgctagttttatttttataacaaatttttaaagtccctgtaaaccctataaaattaactactaatcctaaacttgaactgtaatacatatttttgttaaataaaaaaaaagatatcctaaatttgagaatctaagctaaattcacaaaatgactaaatgtatcttcttactcttaaacgataagaaaccaggaatgcagtggtttaacccaaacagcctaggtattcttcactcatttctcttgtcctcagtatatgtttcccccaggccaagaaacatttatggctaggaattcatgactgctcatctgtaagacactctccaagtactctgtcatgatttggaaccagagctttggccttccattccagatgacctccaggttctaagctttatctgaagtactccacactacattgcacaccagcatgatgggaaggaatgactcaagggaactgggcattcagaccactcaccaactacacacagcctttcacacagcagtagacaacctttgtaggaagacctctggccaaatatttatatatcaggagtatatcaatgtatggtcacagttacctcctttttaatctcaagaaataaagtcattacttctgaaaagaacaccagtaatggaactacacgggcatgccaagatgaaaattagtctctctctctctctccaccccattcttttggccactgcattttaaacaggccatagtgatagaaccagaaggtagctgtgtttcgattatttttgttttcttatttttaaacattgctttatagaagcctgttctataatgtaagaagtgaataggaatcctagtatctggaatgatgctgagccatggagatggccagattccatgtgaagagaagaggctggcagaaagctaagtatttgctcgctttccctaccctgtattcatggagcagccattacaaaagatgacgcaccagaccatgggcctggtcttgctaggtggtggagatccaaaatgctgggcactcactggagctccgtgaagatctgttgaatgagtgactaaatgtgtggtgaactgtccctacctacagggttccctgctttgcccagcctatcaggcttgggtacaagcagatagcctggattggatttcatgagggctttgtgcccactctgtatactaccggaagatgcataatctctttagacttcctgggtcatcactgaaaggccagaggcaagaatttgagcagtaaattgaagcatgtgcagagtgacatagtctgggcatagataacagtacctgacccctcaaattccctaaagttgacatgacagcttgtgcatcatggtttggtacagagatgactgaagagacagtggttgctcctctggttgctggacaccttttgaatgggctgtgatgactggagagcgtgctctgtcctggtcacctgaaggtgctcctagactagcagaagcagagacagcagccaggcctgactggagagctggagggactgggtgcaggggacttctaggccagcagtttctgtgctgtgctcggaggggctctggtggcaggaacctagtcctgggttccagctctaccagggtgcagcctgaggaactcagctttcctatttattggcttgccttgccacaaaggatttcttgtgtgttaaagatactaatagcttaaacaaacaaacaaacaaacaaacaaactattgatttaaaccactaatcaaatctttggttttacaaatttagaaaataaaattaggccccagggactaaatgtctctcctatggtccagaaaccagactgaaactgacctagtagcaaaacccattctcctgtatcccaactcccagttcaggttcctcttcattagctcccacccagctgcatagttgaggacactgaattgaggggacctggggtaaagggctggcactcaggcatcgctggcttctacatgattccaccacatggaaagcatcataatcatgtatgatccctagaaaagtgtttgctaaactccttaagttgtcattcaacaaacatttatctagcacctaccacatgctaggcaaattctagagacacagctgcccctgccctcatggaactaaatgtcttgaaagagatactggccctgatcagattttttaaaaatacataattatgcaaataaatcatattatatataattacaaagcatggtatgaaaggaaaagtacaagatgctgtgaaagcgtataaaaggaggacttggccaagtctagaggatcatcagtgagtgttccctgatggactaacattagggccaagatcagaaagatgtccaggagttaacatggtcaataatcactgttaatctttattaagcatttcccttgagtcgagtcaggcttcacttgctgcattaacttctttaatctccacaataactctgtaaggtaggtgcttttgctttttttccactttacagatgaataaacaagaagtaaaaagatgtaactaattcacatgtagccctctggcttcagaagctctttactccataaggttcctgggcacgtgggtgtggtctaggaatgggaagaggctaagaatgctgaagggcagactgaatgttccagatgaagctggcgtgataagaaagggccacatcatgtgggtcttataggttatgatattcatgttaaaactgttaatgtttttgaaatatctaaaaccaggaagtgcatgatcatatttatgttttagaaagatcactcaggctgctgtgtggagactggagtagaaagaagcatagaagagacctacagagactaggaagctgtcacagtaacctaggcacaagatagtagtggtcaagactagcttgcttgaggaagtgtagagaagtagacagataaaaggagcatccaaggagtgtacacttctaagtaatttaaaagaaagagtcaaatcccttagaagcaatcatttgaaataacctaattaccaatcctttttatccagtcctcttatgttgtcccaattcaggaattcttaaaccagttcttgtgttaaaatcatagcatggactgccttacaacattctatacttcagtccattgaaaaccacatcaggccaggtgcgtggctcatgcctataagcccagcacttttggaggccaaggcgagaggactgcttgagcccaggaattggagaccactgaactctagcctgggcaacagaatgagaccctgttgaaagagagaaaaggaagaaagaaagaaaaaagaggaaggaaggagggaaaaaaggaaaaaagggagggagggaaggtggaagaaagaaaaaaaaagaaagagaaagaaaaggctgcttcctggcacttctattaccatgtgacacgagaagggcaattgtccagacccacacttaggttcaccacagtccttggttcccaggacagagtcctttgaggttccttcaagcacccacaccatgctggaaaatgcaggctttataaactccagatgtcagtccacaactccacagccccattgggtttggttcctggatgaattctcggctagatgactggaatagagttctggtgaacatcttccttcctgccaagatgacaagtcaggtttctaaagacagatgtgtagctctttgtggaaaaaatattctgggcacgaacatcttcggctgaacggcatggaatggtgtggaaaatgtgaagccctggactggtttctgtgtcagtggtttcctgggactgccgagttgaaggggagcagaaagggctcagctgtggaggaggcagagattagccaaactagaccagctatcgccccagctgcatggatagcatcttccttcttgttcctgattaagctcatttaatatgccaatgacaaaaggatcatgaaggtctttccaaatgaatgaatgttttctggagctgagattgctggctcctatttgcatagtagtgcatactgaggcatctgaccggattactggattcctaacttttacattctttttaagctcagatatttctacggaaggcagaaagataaaggccaaaaaaagatcaggcttgcctagctcttgggtttccaggaaactgacagactcctgatatctgcaactagtgctttctctccctgggcggggcgttctccgccaccttgtggacatttggggaactgcaccgccccagcatctatgatcagaggaactgacagaggctgccataactacctggtcccctgaaataaaaaccaggcaccaggtttgcccaaaggaggaccaacacatagcagaatgaggctgagtaggatgttaaccttccatgctggtcttccattatcacgttatctgccactgtcacagacaacatctgcatccgctacagatggagatggaacaaaggctttcactggatctcaagttggatgcttctgtaaaagtaaagttccaagacctttttcttttttttccccagtgacagcaagaaaattggatgttactattggcaacaactatttcctgcttgtgaagcgttggtatttgcagtgcttctgccctgtggttattgagctgaaaggggggctgctcttgacataggagcatggcaggattggattcttcataacatgctacccagcttgtcttatgtgaaaagagaagcccagaaagagcttgggacacaaagatgcaaatcctgggtttgcatttgatttgttaagtgtcctttcacaaccagcttgcagactttaggggaaaaaaaatacatggtaggcttgtgggtagtggagaaaacataagctccagtacctcacagcctgggttttcagtctgcctcctcccaaccctcatcacataccagctgtgtgaccgtgggtactctgtctatgacaatcaattttcttgtctgcagagtacagataatagaggacctgccccatgggaagcttggaggactaaatgagattaagcccagggcctggctcatggccgattctcgtaacaggtccatgtgtgtaatcaacatgtgctgaagttctctggaccctctgtaaatatctaataggaacgactagccatgggtgtgatgagggagatgctagaaacagtaatggggtcaaatgaatctgtcccatagtctctaatttgaaacagctgaactgtgccattgtctccttggaggtaagatataagccacagagtgcctgaagcaggtctacagttaaggaacttaaatgtgctaagtgattaaaggctgggctgcctactccctgtatggcgacactctccaccctcctgcatactctccactactccaggcagtggtgggcctcctagcctctgggtgatgagcagctcccacatctcccctttgagacagaggcctgcagaacctgaccagtgtgagggctggctggggtcaggagtgtgcctgctggttggccctcaaggtgaatgtggctcttctaaaagacacctgaagggatgcagaatttagcagcaaacgccaagaacagctctttgtcatctctgcatcttctactaaagattttaagctccgaagtatatgtctggggtttctagagattgctattcccttataagggaaaaaatacacccttattgtatatttattgagcacctacaatgtgccaggcaccccagctagggagtttagatatttaatcctctcagctgccctgtaaaatagggattgtttaaaatggatttcgcccaccttataggcaaggaaactgaactacagtatggtaaagtggcttggctaatccacctggctagtgagaatggagccacagtgaacctggaggatactcttcacggcgttccatctttgtgtgccccacagcacaagcacaatgcccgggtcttaggaagagcttggtaaatgactgctgagttgagaatggtaggattcatgtaccaaaagggcatttggaaatgggcaaatgcccagatagggctctgaaagactagctatgtagagcaacagatggcaaatcatcaatttaaaactctgtcatccagccagtagcccgctggcctacagccagccatgtatgtacaagggctgacttgatcagctgtggctcttttatttcaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaaggtaagtgaagttcaggggaatgttccaccaatctaaaatttagatggctggatattttgctccttcccatttttttcctttaatgtttcaggaaaagactataatactgttgaagaatttagtttcttccttgagtttgccctcttcatggatagatttaaaacacacccaaatgccaaaagccccacttgcttcttcaagtcttctcagcatcagattccatacatcactgcttcctctgccttttaaaatcttgatcctgacatgccaaagccactctcagcttgtgtttacaggagagataactgaactaaaggggttctaattgggcagagcggggctcaagtcctggccccccaactagaggcagtgtgccttaggcaagttgcccaatctcatgggcctcagtttctttatcaaatgagggagtaccttatttatatgtctgagtctattgcgggatctggccagcagcccgcaatgcaatggggctctctctttgttcctaggcagattggcaggttgagaaataatagacacacacaagatagtgaaagctgggtccaggggggtcactgccttctggtcctgcggtgccaacaatgcactggatataccagcatttattattaagtttagtgagggcaggggtacgttagtgagggatttagggtcatttgattatgaggtgagatggtcacatggggatgaagtaattctttaacataacatttgtatgtagaagtacagtacatttgtatgtagaagtacagtatgagataagaatttacaatatagtgtgtgcgtcagtaatttctaacagagccttagaacagaaacacagtctttccataacctatgattagcaagatattaatcagcagtaacaattgcaacaaaaactggttacaaataatccatggaaacaggacatgaagctagataaccagttagaccagaaattctcagaggggagtacgccttaaccctaaagaggcctagaagagcggcggcaagatgagcgtgtttatagccctatcttatccatatggacaggccccccccatgcgtccgtttataggctccccacaagggtcacattccattcccagagctatgaacatctgcttttctgggataggaatcttggtgatgggaaacctccctgactgcacgtccatttgtaggctctctgcagggggaagcacatcacgtgctgttggctcattgtggcagtccaacctggcattgtctttacacaatcctgcatgcaattttgtatttacaataatcaggagcatttcatcttttattccgtagcaatagtttcagggcgtctccctacatgagtctcagagtaaaaatacatttttatatcattgattttcatatgtgtacagaacacactcctgtgcttgtctctcactttcccttttctgttgactatgatcatgtcaggaagatgaggtgcacagcacccaggtgagcctgtagtcacaacacagccctctcagtagccagatgggaaaggggcccaggaccacactgacagcgatagcccagctgtacctcagcaggccttttataactgcccctgggcctttcagagctcctaccgccaccactaatgctgtcccaggtgggccaatgggtagctcatacagatgtgatgagctggtagatgttcagaatattcttaaattaagatgcattccaggtcttattaccaggaaacaaaatagaagttcctaaggaaatttcatggtcaaaaccaaaatgcacacaaatatggtaaacaaatatgttctcacagcaagtaagatagatgatgtttcattccttttccagaatttagcccagtgtgagatttaattgtagggtaccttcttttctttcttctagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatggtaagtcatcagcaggagtggacatatgctagggaggaaggaaaggaagagggaagaggaaggaaggatgcgagaagggatagatcgatagctacagaatgagggatttcaaaacatttttagccagggtggtggcatgtgcctatagtcccagctactcgggaggcttaggtgggaagatggcttgagcccaggaggctgcagtgagctataattgcaccagtgcactccagcctgggcaaaagagcgagactctgtctctaaaattaaataaatatgaaaaataaataaaacattagtgcctaaacaaaagcctgatgtagcttcatactcagtaccttcttaaggtgctggaaggagatcaggtaaccaccaacatactgctttacaagaactaaaacctttattttaaggtgttcttagatgtctgaattttggatgttgctttccttttgtaagatgatgcaaatgcacaaactctgctaccctggagatatcaacataatagataaaagcatatctgtttggccagactgtttttcactcatctgttcacttgactccagagaaccctggccttgtgtgacaggaacctctttcacaggcacagaaagacaacacacagttttaatggtaataacaagtcctatggctgagcaagaccaggtatgaagaacaagcccgtctacttgtcaccaggcggaatggccatggtagctctctgggggccttactgagccaaggtctcctatgtcggactactacttccatgggctcacagcctgcacccagggaagtcatttctgtcagtgcttggcatgcattgcccagtgcccaggatacaactgcactcgataaatgtttgccgtgtaaatgacaacaatcatgctcatgccaagaccagactcatgatgtctcctcagcatcatgcagtcaatgccagagaagtagttaaggagaagctggtatataaatgttaagagaagtctggagggaaacaacccaggctgttggcagtgcttatccactctgttaaagaggctaggacaaaggagagcttttgctttttatttatttatttatttaattaattaatttatttatttattgagactgagtcttgctctgtcgcccaggctggagtgcagtggcatgatctcagctcactgcaacctccgtcccttgggttcaagcgattctcctgcttcagcctcccaagtagctgggattagaggtgtccactaccacacccagctaatttttgtatctttagtagagacggggtttcaccatgttggctagactggtctcgaactcctgaccttaggtgatccacctgccacagcctcccaaagtgctgggatttacaggtgtgagccactatgcctggccttttttttttttttttttcggctttttgtctcctaaatgagaatatattcattttatttcttataactaaacatatattttgtaaaataaaagtaagaatggatgtgattctccccagcttcaaaaggctgtctctgtctttccaaaattgccaacttgggaatcagctgcagacatagacgtggaggccaggttctctttggcatgaggtctggtgttgagcaggctctagggccaagccctcagaatcaggcagagggcatggcctgccctggagaagcctgtgcaggagtgactctgcaggccaccttgcatgggaggcccagtcccgacaccagggctacaaccgagagaccattttgctaaggccacctggcctgacagtatgggagttttaggagggggaaataagagtttgacatggactggtaacagtatatttttacttctgggagcataaaattagttctaattatagttgagagatccctgggaacacagcagcaggtgaaaagcagctttatttcctcactagagctaaccagattcctggagctgatcctaaggaagaagagccagagtaactagtgtaaatctgtgctactgttgcagattaacaacaacctgagacacacggatgaaatgttctggaaccacgtatccatgtgcattttaaatggaggtggtatccggtcgcccattgatgaacgcaacaatggtatgctcccaggcccagctcctcagtgtgtcatgttctctctctgatctccttttctgttatggttcttgccctgaacacacccatgtgcagcctcagttcatctctgctgccagccatgtaccagaatgtcagtcctttaaattagggttagccattattaaagtctaaaacctagcctgaaggacacacacaagcttgtgtgttcttatatgtattcacacacagaagctgcaggtaaggttagtggaactcaaacttggtctgaatttctgacttacaaagctatactagttgtttctgatttatgcaaaataacaacagtcaccaccttggcatctgaaaagagacaaaatcaccaatcttctgtcctgaaaaagacataccactggattaaaatgttaccttagtatatttacagatatccaagattatatcatatggcaaaacaaatcaactacacaaatactcaagacactcttgtagccctccctgatttactgtgaaaaccaatcagtatcagtataaggccccaaacgcatcccattgtcagacaacagggacaggttgcaggggcacattgtttgtctggaggaacggaacaatgcaaaacaaactactggagatccagccccaagctagcagatctaagactgagcatggtgaacttctgaggcacagccatttgtgagctcccgccatcttgcccagtggtggagtgcagggccagaggcctcctaaactcattcccttcctcagtgtcaggaaccacttagacatagctaatgatgctctataagcttcccatgtccccctcatttcttcccccagcgctgagggaaactggtcatggtgtaagcattttctcaagctattttccttcttgcctcatctgtgactaccctcaggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggtaagtcacccatcctgtagggctggcccatccaaagtgacatggcatttcctgctggttggctcagcttccccttcaccaaaattcctgtggtcaaactggtttggatttttcccgacccaacaccaataccttaccctttaatggctgtggactctcagagccactaagattaggttggggtcaaagccccaaagccccaaagccctggcaataagcagtatttgccaagtctgatttggacatcagatgggccctctaagcaggtcacctgctttgaggtctagtttctaataaaatcttatttaagcattcaattattttttttttaaatcctctcctctgttctactcatcgttgcctggaaccatgccaagatttaaaggaatgctgtccaatctgtattaaccaggatgattcctggaaggagcagcctactgcacagacatacctgaacacctggttctagtcccactgtgacctggggcaagttctcaacccctctgggcacagggagaaaacctttcctgtttatctcatggactgcagtgaaagttgaatggaaataaatgcttcctaaattgccaagggcttctgaaacacttttgttaataattccacagtccagtgaattaatcctttaaaagcaatcttattttacctggaaatcataatctggcctcatttttttaaccttaggttgtggtcccgcttgacagatagaattgctttcctgagttttcctctgcagaacacattgctgttgagactgaagggctacagaaggcagtggggccctgtcctaatgcctatggggcaacccaaacatcctccattttcccagaagggagttgagggaaaagcagttttctgatgctctagcaaagcactgcatagctctatgccttgtggaagtcaccaaatcccaaatgattaccatttaatggtcttcaaaattgtaaaatgctctctgcctctgatctgtttctcactttctaaggccattgaatcaatttagtagaccaattcagcattttaagggaaaaacagaaaaagatgtggttttgtccatgtagagtcaccctttgcctattctggtccagagctggttttccttccagtggttctcatatgtcctgtaaggactcaaggataaagggtggctgcaatgtcacaattctcttgacaaaatttcactcaggtttaaaccaaaggaaaaaccacagccccatgccccaattgtagagtttggccaaaattccttaggaaacagaaatctccctttggatctggtgaaaacagattcatttcttttctaggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtaagcatgactgtctcttcctttctctaaagaacaacaaaattgggccaagaaggggagctactagtggtgccaggagggcaaagtgactccctgtattgctgactccctcctgctttgcttgttggaaagcagcagcacagcacagcagagaggaatatgtaccctgtcaatccccgagcacacacatgccttcatccaaccccagagacaaaacatttgtaaatcaaaaatgttttaaaattcttttaactacatctaagcagctatttaaattgtgaaatataagtgagttttgcattaacagaaatatcacacctctggaattttaggatatggaaaaccatcacattaaatcatttaaattttctttcttgaagaagtttctgtgtgaatcagtcaggtagaagtggatggggtgagcgccgccctttcagcacagtgggttggtggtacaaatcataggaataaccatacttgatgtcagttggtacttctggcagcattgagggacattatatgccagcctcacttttagaaattctgttgagagtcacattatgtgcatacagaccactgacataggacacacttccccttcctcttcctagggcagagaccagcatgcttacaagacacacaagcagcttcttggcatctctgggtagttccaccaggatcttcctcagtcttatcatgctggacctgtcattttagctacttattccaaggagcgtggagggcaggaccagaccaaaggaggcagcccttcctgggaggaaaggtgctggcttcttctgtcccttccgtgacagaagatggaagggcaggcgtaaaagccccatgagacaaagaggctgatagccatcagctgtccaccactcaagcctagactgaaggatgttagagagaggagctcatgcagaaggctaaggaagccctggacctaagcgttctcaacactggttatacattagaatcacttcaggagcattttaaattaaataccaatggctcggccgcagctcttaagagattctaagttaactggattggggctaaactctttttcagtgattctagtgtacacccagaggtgaaaaccactgaagagagaagggttcttaagtactggagtgatatttttaaaataaaattctgttgggttcccaacagaacatgggttcccaacaaacatgggtttgttgtagctataggcaattagcatttatcctctcctctctttggtaaaggaaatcccccaagtcccatcatcaattacatgaaaataattattgggcaggcaaacaaaaggaagccagttttatctctcaactaattgctcttgacccagttccctaatgcccttgttctggagtggttcaaaggctatgatgtcaatgacctctttctagagtcatcacagagctgaggcaactctacttcccccaattacccaatattgaacaacagacacattttacatctggagcatgaaaaagcaatcaaattctttgtacttatgatcactagcgttcttgtcttctgtgacactttacccctgcttatgaaattgcttcccttttataattacaaaggactaccttactgttgattgataattaccttttctgtagttaaaataatgtatatgtttttctaggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatagccaaaaattctccttgcctttaatgtgtgaaactgcattttttcaagtgagattcaaatctgccttttaggacctggctttgtgacagcaaaaaccatctttacaggctcctagaagctgaaggttagagcattataaaatgaagagacagacatgattactcagggtcagcaacctagtgagttagaaaaaaaattaacatagggccctataaggagaaagccaactatgttaagtttacgtgtccaaattttaatgaaattttactaacaattttaaaccatatttttcttcttcatatccatttctaatccatcaaacagcttatgtttacataaaattttatcattcacaaggaagttttaagcacactgtctcatttgatatccacaacttatttttggtaggaaagagagatgtttttcccacctgtcagatgaaaaaactgaagctcaaaaagggttgacttgaccatacagctaatgctgacagatccaagacctagacctaggtcttttgaactcaagtccagcattctcaactatatcaagttactgttcagaatacttaatatctcctctcttcataattatcaatagccccaagctcatggatgacaaatctctgctttatttcttgtctctattttttcactttatagctcctgttataatagcaagtttaatggtataaacacaggataccatcctctcttgcaacacccatgtgcctttgatgagtcaggtagcaagctgtagtagataatgagaaaggccagaggctgcaaaagacagtcaaaggacacgagagaaaggaaggggaagaacaggactccaggactgttttatattatagaaaagcaagagctaaagagcatttacacatgttaaacagatacttgttaagcatagtgcctgacacacggcattagctgttattttatgagattccatcagctctgcctctgtcctctttcttctaacatgaaggtatcatgagaagagaaccttctaacataagctgtaattctaaacctgcacttgtccctctccagcaagaggctagcactgaattcattctactcatactacacacccagttatggaatgtccagagttctcgaagaaaataaatgactttaggaagaggtatacattttttaagtcgctctgcctccaaatctgaacagtcactgtaaatcattcttaagcccagatatgagaacttctgctggaaagtgggaccctctgagtgggtggtcagaaaatacccatgctgatgaaatgacctatgcccaaagaacaaatacttaacgtgggagtggaaccacatgagcctgctcagctctgcataagtaattcaagaaatgggaggcttcaccttaaaaacagtgtgcaaatggcagctagaggttttgataggaagtatgtttgtttcttagtgtttacaaatattaagtactcttgatacaaaatatacttttaaacttcataacctttttataaaagttgttgcagcaaaataatagcctcggttctatgcatatatggattagctataaaaaatgtcaataagattgtacaaggaaaattagagaaagtcacatttagggtttattttttacacttggccagtaaaatagggtaaatcctattagaattttttaaagaactttttttaagtttcctaaatctgtgtgtgtattgtgaagtggtataagaaatgactttgaaccactttgcaattgtagattcccaacaataaaattgaagataagctc

SEQ ID NO: 3 - DNA, 16 bp, Artificial Sequence
  oligonucleotide
gcgagtgccggcgagt

SEQ ID NO: 4 - DNA, 17 bp, Artificial Sequence
  antisense oligonucleotide
agctgtggcgcgtgaac

SEQ ID NO: 5 - DNA, 17 bp, Artificial Sequence
  antisense oligonucleotide
gctggcgttgacgcact

SEQ ID NO: 6 - DNA, 17 bp, Artificial Sequence
  antisense oligonucleotide
cctggtactggtcgccg

SEQ ID NO: 7 - DNA, 17 bp, Artificial Sequence
  antisense oligonucleotide
ccgtgtgtctcaggttg

SEQ ID NO: 8 - DNA, 17 bp, Artificial Sequence
  antisense oligonucleotide
cacgctatgctcaaagg

SEQ ID NO: 9 - DNA, 17 bp, Artificial Sequence
  antisense oligonucleotide
tcatacaccacatggat

SEQ ID NO: 10 - DNA, 17 bp, Artificial Sequence
  antisense oligonucleotide
ggcactcgacacttggt

SEQ ID NO: 11 - DNA, 17 bp, Artificial Sequence
  antisense oligonucleotide
cttatatacctcgtcca

SEQ ID NO: 12 - DNA, 17 bp, Artificial Sequence
  antisense oligonucleotide
gtagaaaccacgttgat

SEQ ID NO: 13 - DNA, 17 bp, Artificial Sequence
  antisense oligonucleotide
gctgtatggtcaagtca

SEQ ID NO: 14 - DNA, 17 bp, Artificial Sequence
  antisense oligonucleotide
ctcgtgtcctttgactg

SEQ ID NO: 15 - DNA, 17 bp, Artificial Sequence
  antisense oligonucleotide
tagaaccgaggctatta

SEQ ID NO: 16 - DNA, 18 bp, Artificial Sequence
  antisense oligonucleotide
ggacacatagctgtggcg

SEQ ID NO: 17 - DNA, 18 bp, Artificial Sequence
  antisense oligonucleotide
ctgtgcacgtcgttggtg

SEQ ID NO: 18 - DNA, 18 bp, Artificial Sequence
  antisense oligonucleotide
tggagtcctcgctggtct

SEQ ID NO: 19 - DNA, 18 bp, Artificial Sequence
  antisense oligonucleotide
ttgacgcacttgctggag

SEQ ID NO: 20 - DNA, 18 bp, Artificial Sequence
  antisense oligonucleotide
atggcatcgtagcgcagg

SEQ ID NO: 21 - DNA, 18 bp, Artificial Sequence
  antisense oligonucleotide
ggaaatttggcctctttg

SEQ ID NO: 22 - DNA, 18 bp, Artificial Sequence
  antisense oligonucleotide
aagtgtatccaacgattc

SEQ ID NO: 23 - DNA, 18 bp, Artificial Sequence
  antisense oligonucleotide
atctacttcaggttgtaa

SEQ ID NO: 24 - DNA, 18 bp, Artificial Sequence
  antisense oligonucleotide
tttgttcacatttagagt

SEQ ID NO: 25 - DNA, 18 bp, Artificial Sequence
  antisense oligonucleotide
ctttctgagcgatgagtt

SEQ ID NO: 26 - DNA, 18 bp, Artificial Sequence
  antisense oligonucleotide
gtggattgcctgtgtaaa

SEQ ID NO: 27 - DNA, 18 bp, Artificial Sequence
  antisense oligonucleotide
ctacaggaaccttccgcc

SEQ ID NO: 28 - DNA, 18 bp, Artificial Sequence
  antisense oligonucleotide
gcataggcctggactaca

SEQ ID NO: 29 - DNA, 18 bp, Artificial Sequence
  antisense oligonucleotide
ttcagatagcctaggtat

SEQ ID NO: 30 - DNA, 18 bp, Artificial Sequence
  antisense oligonucleotide
aactcgatcttcagatag

SEQ ID NO: 31 - DNA, 18 bp, Artificial Sequence
  antisense oligonucleotide
ctctttcatcaaactcga

SEQ ID NO: 32 - DNA, 18 bp, Artificial Sequence
  antisense oligonucleotide
ttatgcttggatcttcag

SEQ ID NO: 33 - DNA, 18 bp, Artificial Sequence
  antisense oligonucleotide
aggagccatccagataga

SEQ ID NO: 34 - DNA, 18 bp, Artificial Sequence
  antisense oligonucleotide
ggataccacctccattta

SEQ ID NO: 35 - DNA, 18 bp, Artificial Sequence
  antisense oligonucleotide
aggtaattgtgccattgt

SEQ ID NO: 36 - DNA, 18 bp, Artificial Sequence
  antisense oligonucleotide
gtggaaccttttaactgg

SEQ ID NO: 37 - DNA, 18 bp, Artificial Sequence
  antisense oligonucleotide
agtggactggccgtagcg

SEQ ID NO: 38 - DNA, 18 bp, Artificial Sequence
  antisense oligonucleotide
cgacacttggtgcaaaga

SEQ ID NO: 39 - DNA, 18 bp, Artificial Sequence
  antisense oligonucleotide
atacctcgtccattttga

SEQ ID NO: 40 - DNA, 18 bp, Artificial Sequence
  antisense oligonucleotide
cgaccttcaactgctgga

SEQ ID NO: 41 - DNA, 18 bp, Artificial Sequence
  antisense oligonucleotide
aacttgatccgaccttca

SEQ ID NO: 42 - DNA, 18 bp, Artificial Sequence
  antisense oligonucleotide
cctgtggaaaacttgatc

SEQ ID NO: 43 - DNA, 18 bp, Artificial Sequence
  antisense oligonucleotide
ggtcctaaaaggcagatt

SEQ ID NO: 44 - DNA, 18 bp, Artificial Sequence
  antisense oligonucleotide
acgtaaacttaacatagt

SEQ ID NO: 45 - DNA, 18 bp, Artificial Sequence
  antisense oligonucleotide
tcagcattagctgtatgg

SEQ ID NO: 46 - DNA, 18 bp, Artificial Sequence
  antisense oligonucleotide
ggatctgtcagcattagc

SEQ ID NO: 47 - DNA, 18 bp, Artificial Sequence
  antisense oligonucleotide
gatttgtcatccatgagc

SEQ ID NO: 48 - DNA, 18 bp, Artificial Sequence
  antisense oligonucleotide
actcatcaaaggcacatg

SEQ ID NO: 49 - DNA, 18 bp, Artificial Sequence
  antisense oligonucleotide
attatctactacagcttg

SEQ ID NO: 50 - DNA, 18 bp, Artificial Sequence
  antisense oligonucleotide
ataacagctaatgccgtg

SEQ ID NO: 51 - DNA, 18 bp, Artificial Sequence
  antisense oligonucleotide
gcttatgttagaaggttc

SEQ ID NO: 52 - DNA, 18 bp, Artificial Sequence
  antisense oligonucleotide
tcgagaactctggacatt

SEQ ID NO: 53 - DNA, 18 bp, Artificial Sequence
  antisense oligonucleotide
tggaggcagagcgactta

SEQ ID NO: 54 - DNA, 18 bp, Artificial Sequence
  antisense oligonucleotide
ggcataggtcatttcatc

SEQ ID NO: 55 - DNA, 16 bp, Artificial Sequence
  antisense oligonucleotide
cgagagtatgctacca

SEQ ID NO: 56 - DNA, 16 bp, Artificial Sequence
  antisense oligonucleotide
tgcggccgagccattg

SEQ ID NO: 57 - DNA, 17 bp, Artificial Sequence
  antisense oligonucleotide
gaatcaatatgcggtga

SEQ ID NO: 58 - DNA, 17 bp, Artificial Sequence
  antisense oligonucleotide
gtaacaaacgatagcct

SEQ ID NO: 59 - DNA, 17 bp, Artificial Sequence
  antisense oligonucleotide
tggttgcaaactgtgag

SEQ ID NO: 60 - DNA, 17 bp, Artificial Sequence
  antisense oligonucleotide
gtagtccgacataggag

SEQ ID NO: 61 - DNA, 17 bp, Artificial Sequence
  antisense oligonucleotide
ttagatctgctagcttg

SEQ ID NO: 62 - DNA, 17 bp, Artificial Sequence
  antisense oligonucleotide
gagccattggtatttaa

SEQ ID NO: 63 - DNA, 18 bp, Artificial Sequence
  antisense oligonucleotide
cacactctgccatccgct

SEQ ID NO: 64 - DNA, 18 bp, Artificial Sequence
  antisense oligonucleotide
gatctggtgtccattctt

SEQ ID NO: 65 - DNA, 18 bp, Artificial Sequence
  antisense oligonucleotide
tagctgtggaataccaat

SEQ ID NO: 66 - DNA, 18 bp, Artificial Sequence
  antisense oligonucleotide
gccatctaaccttgccat

SEQ ID NO: 67 - DNA, 18 bp, Artificial Sequence
  antisense oligonucleotide
atcaatatgcggtgagtg

SEQ ID NO: 68 - DNA, 18 bp, Artificial Sequence
  antisense oligonucleotide
ggctcctttgaactaggt

SEQ ID NO: 69 - DNA, 18 bp, Artificial Sequence
  antisense oligonucleotide
ctagaaagtgtacacctc

SEQ ID NO: 70 - DNA, 18 bp, Artificial Sequence
  antisense oligonucleotide
cctacaataaagctggat

SEQ ID NO: 71 - DNA, 18 bp, Artificial Sequence
  antisense oligonucleotide
agaagtgaattgcatagc

SEQ ID NO: 72 - DNA, 18 bp, Artificial Sequence
  antisense oligonucleotide
cagaggtaagctggttca

SEQ ID NO: 73 - DNA, 18 bp, Artificial Sequence
  antisense oligonucleotide
atgccaagctgtgattta

SEQ ID NO: 74 - DNA, 18 bp, Artificial Sequence
  antisense oligonucleotide
ctgtttagcactggctat

SEQ ID NO: 75 - DNA, 18 bp, Artificial Sequence
  antisense oligonucleotide
gttacagcctggtaaagg

SEQ ID NO: 76 - DNA, 18 bp, Artificial Sequence
  antisense oligonucleotide
atgtcgagagtatgctac

SEQ ID NO: 77 - DNA, 18 bp, Artificial Sequence
  antisense oligonucleotide
agatccagacgttcttac

SEQ ID NO: 78 - DNA, 18 bp, Artificial Sequence
  antisense oligonucleotide
ggaggcttcagatattgt

SEQ ID NO: 79 - DNA, 18 bp, Artificial Sequence
  antisense oligonucleotide
aaggtggaaccagattca

SEQ ID NO: 80 - DNA, 18 bp, Artificial Sequence
  antisense oligonucleotide
tggaactttgagcatgat

SEQ ID NO: 81 - DNA, 18 bp, Artificial Sequence
  antisense oligonucleotide
cttaagtgaaggccaact

SEQ ID NO: 82 - DNA, 18 bp, Artificial Sequence
  antisense oligonucleotide
aatccacgagctttggaa

SEQ ID NO: 83 - DNA, 18 bp, Artificial Sequence
  antisense oligonucleotide
gactctaggatttaactt

SEQ ID NO: 84 - DNA, 18 bp, Artificial Sequence
  antisense oligonucleotide
ccgcaatagactcagaca

SEQ ID NO: 85 - DNA, 18 bp, Artificial Sequence
  antisense oligonucleotide
acgctcatcttgccgccg

SEQ ID NO: 86 - DNA, 18 bp, Artificial Sequence
  antisense oligonucleotide
catctggctactgagagg

SEQ ID NO: 87 - DNA, 18 bp, Artificial Sequence
  antisense oligonucleotide
attagtggtggcggtagg

SEQ ID NO: 88 - DNA, 18 bp, Artificial Sequence
  antisense oligonucleotide
ggtactgagtatgaagct

SEQ ID NO: 89 - DNA, 18 bp, Artificial Sequence
  antisense oligonucleotide
gtagcagagtttgtgcat

SEQ ID NO: 90 - DNA, 18 bp, Artificial Sequence
  antisense oligonucleotide
gtagtccgacataggaga

SEQ ID NO: 91 - DNA, 18 bp, Artificial Sequence
  antisense oligonucleotide
ttggcatgagcatgattg

SEQ ID NO: 92 - DNA, 18 bp, Artificial Sequence
  antisense oligonucleotide
gataagcactgccaacag

SEQ ID NO: 93 - DNA, 18 bp, Artificial Sequence
  antisense oligonucleotide
aatggtctctcggttgta

SEQ ID NO: 94 - DNA, 18 bp, Artificial Sequence
  antisense oligonucleotide
ccagtccatgtcaaactc

SEQ ID NO: 95 - DNA, 18 bp, Artificial Sequence
  antisense oligonucleotide
gatttacactagttactc

SEQ ID NO: 96 - DNA, 18 bp, Artificial Sequence
  antisense oligonucleotide
tttaatccagtggtatgt

SEQ ID NO: 97 - DNA, 18 bp, Artificial Sequence
  antisense oligonucleotide
cttagatctgctagcttg

SEQ ID NO: 98 - DNA, 18 bp, Artificial Sequence
  antisense oligonucleotide
tattagaaactagacctc

SEQ ID NO: 99 - DNA, 18 bp, Artificial Sequence
  antisense oligonucleotide
atgcagtgctttgctaga

SEQ ID NO: 100 - DNA, 18 bp, Artificial Sequence
  antisense oligonucleotide
cacaaggcatagagctat

SEQ ID NO: 101 - DNA, 18 bp, Artificial Sequence
  antisense oligonucleotide
gtcagtggtctgtatgca

SEQ ID NO: 102 - DNA, 18 bp, Artificial Sequence
  antisense oligonucleotide
ttgtaagcatgctggtct

SEQ ID NO: 103 - DNA, 18 bp, Artificial Sequence
  antisense oligonucleotide
gagccattggtatttaat

SEQ ID NO: 104 - DNA, 18 bp, Artificial Sequence
  antisense oligonucleotide
gataaatgctaattgcct

SEQ ID NO: 105 - DNA, 18 bp, Artificial Sequence
  antisense oligonucleotide
ttgaaccactccagaaca

SEQ ID NO: 106 - DNA, 18 bp, Artificial Sequence
  antisense oligonucleotide
ggtagtcctttgtaatta

SEQ ID NO: 107 - DNA, 17 bp, Artificial Sequence
  antisense oligonucleotide
accaatgcttaagagac

SEQ ID NO: 108 - DNA, 17 bp, Artificial Sequence
  antisense oligonucleotide
atcaatatgcggtgagt

SEQ ID NO: 109 - DNA, 17 bp, Artificial Sequence
  antisense oligonucleotide
ctaggaatcaatatgcg

SEQ ID NO: 110 - DNA, 17 bp, Artificial Sequence
  antisense oligonucleotide
tggtaacaaacgatagc

SEQ ID NO: 111 - DNA, 17 bp, Artificial Sequence
  antisense oligonucleotide
cggtgaaccagatagtg

SEQ ID NO: 112 - DNA, 17 bp, Artificial Sequence
  antisense oligonucleotide
gtgtatccaacgattcc

SEQ ID NO: 113 - DNA, 17 bp, Artificial Sequence
  antisense oligonucleotide
gcgatgagtttatccat

SEQ ID NO: 114 - DNA, 17 bp, Artificial Sequence
  antisense oligonucleotide
accttatatacctcgtc

SEQ ID NO: 115 - DNA, 18 bp, Artificial Sequence
  negative control
cgtttaggctatgtactt

SEQ ID NO: 116 - DNA, 16 bp, Artificial Sequence
  control
gcactcgacacttggt

(19) United States
(12) Patent Application Publication
(10) Pub. No.: US 2024/0076673 A1
(43) Pub. Date: Mar. 7, 2024

(54) Oligonucleotides reducing the amount of CD73 mRNA and CD73 protein expression

(71) Applicant: Secarna Pharmaceuticals GmbH & Co. KG, Marburg (DE)

(72) Inventors: Frank JASCHINSKI, Puchheim (DE); Sven MICHEL, Bernried (DE); Richard KLAR, München (DE); Julia FESTAG, Jesenwang (DE)

(21) Appl. No.: 18/270,567

(22) PCT Filed: Dec. 31, 2021
(86) PCT No.: PCT/EP2021/087892

§ 371 Date: Jun. 30, 2023

(30) Foreign Application Priority Data
Dec. 31, 2020 (EP) 20217955.2

(51) Int. Cl. C12N 15/113; A61K 31/7125; A61K 45/06

(52) U.S. Cl. CPC C12N 15/1137; A61K 31/7125; A61K 45/06; C12N 2310/11; C12N 2310/315; C12N 2310/321

(57) ABSTRACT

The present invention refers to immunosuppression-reverting oligonucleotides comprising 12 to 18 nucleotides, wherein at least one of the nucleotides is modified, and the oligonucleotide hybridizes with an hybridizing active region of the nucleic acid sequence of an ectoenzyme (CD73) of SEQ ID NO.1 (human) and/or SEQ ID NO.2 (human). The invention is further directed to a pharmaceutical composition comprising such oligonucleotide. The oligonucleotide and the pharmaceutical composition are used in a method of preventing and/or treating a disorder, where a CD73 imbalance is involved.

The present invention refers to an oligonucleotide hybridizing with a nucleic acid sequence of an ectoenzyme (NT5E or CD73) and to a pharmaceutical composition comprising such oligonucleotide and a pharmaceutically acceptable carrier, excipient and/or dilutant. The present invention further comprises the use of the oligonucleotide and/or pharmaceutical composition in a method of preventing and/or treating an autoimmune disorder, an immune disorder, a psychiatric disorder and/or cancer.

### TECHNICAL BACKGROUND

In recent years the treatment of several different diseases such as malignant tumors was very successful by application of immune therapy, in particular by inhibitors of so called “immune checkpoints”. These checkpoints are molecules in the immune system that either turn up (co-stimulatory molecules) or down a signal. The concept of the therapeutic approach is based on the activation of endogenous anti-tumor immune reactions. Many cancers for example protect themselves from the immune system by inhibiting e.g. T cell and NK cell activity, respectively. Immune checkpoint modulators, i.e., stimulators or inhibitors are for example directed to one or more of CTLA-4, PD-1, PD-L1, LAG-3, VISTA, A2AR, BTLA, IDO, CD39, CD73, STAT3, TDO2, TIM-3, MICA, NKG2A, KIR, TIGIT, TGF-beta, Ox40, GITR, CD27, CD160, 2B4 and 4-1BB.

CD73 needs to be considered as a novel and promising candidate to improve immunity towards different types of cancers. CD73 is an ectoenzyme (NTPdase) and catalyzes the conversion of AMP to immunosuppressive adenosine. CD73 acts in concert but downstream of CD39, known to convert ATP to ADP and ADP to AMP. Adenosine exerts its effects via adenosine receptor A1, adenosine receptor A_{2A}, adenosine receptor A_{2B} and adenosine receptor A₃. In addition metabolites derived from Adenosine have additional immunosuppressive functions. The range of immune cells modulated by Adenosine or its metabolites include T lymphocytes, natural killer (NK) cells, NKT cells, macrophages, DCs, neutrophils, mast cells and B cells.

CD73 is found in most tissues and many cell types including subsets of lymphocytes, macrophages, dendritic cells, endothelial cells and epithelial cells. Hypoxia induces CD73 mRNA and protein expression and increases CD73 activity in microvascular endothelial cells. Particularly, CD73 is highly expressed in many different human (solid and hematologic) tumors, and its elevated expression and activity are associated with tumor invasiveness and metastasis and with shorter patient survival. The RNA expression and enzyme activity of CD73 are variable in different breast cancer cell lines.

Dying cancer cells release ATP to the extracellular space in the tumor microenvironment. Living tumor cells express often high levels of CD39 and CD73 and convert ATP to the immunosuppressive adenosine and additional immunosuppressive downstream metabolites of Adenosine. By this, tumor cells are competent to perform uncontrolled proliferation and expansion. For example, T cells are inhibited in their proliferation, cytotoxic cytokine production and activation. NK cells show reduced cytotoxic potential. Adenosine induces alternative activation in macrophages (immune suppressive M2 phenotype) resulting in reduced pro-inflammatory cytokine production but increased generation of the immunosuppressive cytokine IL-10. Furthermore, downstream metabolites of adenosine e.g. deoxy-ATP can lead to inhibition of proliferation of effector immune cells, e.g. T cells. The important role of CD73 as relevant therapeutic target in different tumors is underlined by the fact that tumor models using CD73 or A_{2A} receptor knockout mice reveal improved disease outcome.

Anti-human CD73 monoclonal antibodies such as anti-CD73-antibodies of Innate Pharma are currently under clinical investigation. However, because low tissue penetration due to their large size, monoclonal antibodies against CD73 might fail to reach their targets sufficiently. Furthermore, CD73 small molecule inhibitors are currently under clinical investigation, e.g. AB680 (Arcus Biosciences). However, CD73 has been described to have functions in addition to its enzymatic activity, e.g. mediation of invasive and metastatic properties of cancers. It is therefore not assured that a CD73 blocking antibody or a CD73 small molecule inhibitor prevents all tumor promoting functions of CD73.

Immune therapies have resulted in long-term remission, but only of small patient groups so far. The reason may be that numerous immune checkpoints and optionally further immunosuppressive mechanisms are involved in the interaction between for example the immune system and the tumor cells. The combination of immune checkpoints and potential other mechanisms may vary depending on the tumor and individual conditions of a subject to escape the body's defenses.

For the inhibition of several immunosuppressive mechanisms common approaches using an antibody and/or a small molecule are not or hardly suitable as the molecular target is located intracellularly or does not have enzymatic activity. Accordingly, an agent which is safe and effective in inhibiting the function of an “immune checkpoint” such as CD73 is an important addition for the treatment of patients suffering from diseases or conditions affected for example by the activity of this enzyme.

WO2018065627A1 describes immuno-suppression reverting oligonucleotides inhibiting the expression of CD73 protein. A continuous attempt in the field of oligonucleotides is the increase of the efficiency, i.e., the reduction of the amount of mRNA and the reduction of the expression of CD73 protein, and the parallel reduction of undesired side effects such as toxicity.

The mode of action of an oligonucleotide differs from the mode of action of an antibody or small molecule, and oligonucleotides are highly advantageous regarding for example
(i) the penetration of tumor tissue in solid tumors,
(ii) the blocking of multiple functions and activities, respectively, of a target,
(iii) the combination of oligonucleotides with each other or an antibody or a small molecule, and
(iv) the inhibition of intracellular effects which are not accessible for an antibody or inhabitable via a small molecule.

Oligonucleotides of the present invention are very successful in the reduction of the amount of CD73 mRNA and the reduction of CD73 protein expression, respectively, and have surprisingly no or at most very low toxic side effects. Therefore, targeting CD73 protein expression using an oligonucleotide of the present invention is a promising approach to develop and improve for example immunotherapies against different cancers and immune diseases, respectively, resulting in extremely low negative such as toxic side effects.

### SUMMARY

The present invention refers to an oligonucleotide comprising 12 to 20 nucleotides, wherein at least one of the nucleotides is modified, and the oligonucleotide hybridizes with a nucleic acid sequence of an ectoenzyme (CD73) pre-mRNA of SEQ ID NO.2 in a hybridizing active region selected from the group shown in Table 2 and a combination thereof. The modified nucleotide is for example selected from the group consisting of a bridged nucleic acid such as LNA, cET, ENA, 2′Fluoro modified nucleotide, 2′O-Methyl modified nucleotide and combinations thereof. The oligonucleotide of the present invention reduces the amount of CD73 mRNA or the level of CD73 protein expression for at least 40% compared to an untreated control. Oligonucleotides of the present invention are for example shown in Table 1

The oligonucleotide of the present invention reduces the amount of CD73 mRNA or the level of CD73 protein expression for example at a nanomolar concentration.

The present invention is further directed to a pharmaceutical composition comprising an oligonucleotide of the present invention and a pharmaceutically acceptable carrier, excipient and/or dilutant. The pharmaceutical composition further comprises for example an antitumor active agent such as a chemotherapeutic (e.g., platinum, gemcitabine), an immune stimulating agent, disease specific agent or an agent that reverses tumor- or infection-mediated immunosuppression which are for example another oligonucleotide, an antibody, a carbohydrate-modified antibody, a peptide-based therapeutic, a protein-based therapeutic, a lipid, a therapeutic vaccine, a HERA fusion protein, a ligand trap, a Fab fragment, a nanobody, a BiTe, a DARPin, a small molecule or a combination thereof. The antitumor active agent, the disease specific agent such as the other oligonucleotide, the antibody, the carbohydrate-modified antibody, the peptide-based therapeutic, the protein-based therapeutic, the lipid, the therapeutic vaccine, the HERA fusion protein, the ligand trap, the Fab fragment, the nanobody, the BiTe, the DARPin and/or the small molecule inhibit for example expression or activity of an immune suppressive factor selected from the group consisting of IDO1, IDO2, CTLA-4, PD-1, PD-L1, LAG-3, VISTA, A2AR, CD39, CD73, STAT3, TDO2, TIM-3, TIGIT, TGF-beta, BTLA, MICA, NKG2A, KIR, CD160, MTDH, Xbp1, Chop and a combination thereof, or stimulate expression or activity of an immune stimulatory factor selected from the group consisting of 4-1BB, Ox40, KIR, GITR, CD27, 2B4 and a combination thereof.

Alternatively or in addition, the antitumor active agent, the disease specific agent such as the other oligonucleotide, the antibody, the carbohydrate-modified antibody, the peptide-based therapeutic, the protein-based therapeutic, the lipid, the therapeutic vaccine, the HERA fusion protein, the ligand trap, the Fab fragment, the nanobody, the BiTe, the DARPin and/or the small molecule inhibits for example expression or activity of a factor involved in cancer progression and/or metastasis selected from the group consisting of SND1, MTDH, HER-2, BRAF, KRAS, VEGF, EGFR1, EGFR2, BCR/ABL, ABL, MET, ALK, JAK2, BTK, miR-223, CCL18, CCL20, Lcn2, CCL5/CCR9, DDR2, PHD2, IL6, SDF-1/CXCL12 and a combination thereof.

The oligonucleotide and the pharmaceutical composition of the present invention, respectively, is for use in a method of preventing and/or treating a disorder, where a CD73 imbalance is involved. The disorder is for example an autoimmune disorder, an immune disorder, a psychiatric disorder and/or cancer. The cancer is for example breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomater tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythemia vera, adenocarcinoma, anaplastic astrocytoma, glioblastoma multiforme, leukemia, or epidermoid carcinoma.

The oligonucleotide or the pharmaceutical composition for use according to the present invention is for example administered locally or systemically.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### DESCRIPTION OF FIGURES

FIGS. 1A and 1B depict a target knockdown efficacy screen of human CD73-specific ASOs in EFO-21 (FIG. 1A) and MDA-MB 231 (FIG. 1B) cells.

FIG. 2 shows dose-dependent CD73 mRNA knockdown by selected CD73 ASOs in EFO-21 cells after 3 days ASO treatment.

FIG. 3 depicts in vivo assessment of liver toxicity of selected ASOs of the present invention in comparison to an ASO of the prior art.

### DETAILED DESCRIPTION

The present invention provides oligonucleotides which hybridize with mRNA and/or pre-mRNA sequences of the ectonucleotidase CD73 for example of human origin. These oligonucleotides hybridize with an intron and/or an exon and/or an exon-exon junction and/or an exon-intron junction of the CD73 pre-mRNA for example in a tumor cell or a cell in the tumor microenvironment such as an immune cell. Via recruitment of RNase H the pre mRNA is degraded and the levels of CD73 mRNA are reduced. As a consequence the production of CD73 protein is prevented and levels of CD73 protein are reduced to the amount of CD73 mRNA and CD73 protein expression, respectively, for example on a tumor cell or a tumor-associated immune cell. In consequence, the level of immunosuppressive adenosine and its downstream metabolites decreases. All these effects result in an increase of antitumoral immune cells, immune activation (e.g., via cytotoxic T cells or NK cells) and recognition and elimination of tumor cells, respectively, without or only very low toxic side effects. Furthermore, reduction of CD73 protein levels leads to prevention of tumor cell invasion and metastasis mediated by CD73. Thus, the oligonucleotides of the present invention represent a promising and highly efficient tool for use in a method of preventing and/or treating disorders, where the CD73 protein expression and activity, respectively, is involved in disease development and progression.

The oligonucleotides of the present invention hybridize for example with CD73 mRNA of SEQ ID NO.1 (RefSeq ID NM_002526.4) and/or pre-mRNA of SEQ ID NO.2 (GRCh38.p13:6:85450083:85495784).

In the following, the elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

Oligonucleotides of the present invention are for example antisense oligonucleotides consisting of or comprising 10 to 25 nucleotides, 10 to 15 nucleotides, 15 to 20 nucleotides, 12 to 18 nucleotides, or 14 to 17 nucleotides. The oligonucleotides for example consist of or comprise 10, 11, 12, 13, 14, 15, 16, 17, 18, 19, 20 or 25 nucleotides. The oligonucleotides of the present invention comprise at least one nucleotide which is modified. The modified nucleotide is for example a bridged nucleotide such as a locked nucleic acid (LNA, e.g., 2′,4′-LNA), cET, ENA, a 2′Fluoro modified nucleotide, a 2′O-Methyl modified nucleotide or combinations thereof. The oligonucleotide of the present invention comprises nucleotides having for example one or more, two or more, three or more or four or more of the same or different modifications. Further, the oligonucleotide of the present invention comprises optionally a modified phosphate backbone, wherein the phosphate is for example a phosphorothioate or methylphosphonate or a combination thereof.

Reducing according to the present invention includes inhibiting an effect such as expression in different percentages and amounts, respectively.

The concept of the present invention is the provision of an oligonucleotide such as an antisense oligonucleotide mediating the limitation of available CD73 mRNA for protein expression. In order to limit protein expression, the oligonucleotide requires the presence of a complementary nucleic acid sequence representing a hybridization target which allows the formation of heteroduplexes. The oligonucleotides of the present invention hybridize with RNAs of SEQ ID NO.1 and/or pre-mRNAs of SEQ ID NO.2. The formation of a heteroduplex between the oligonucleotide and the target RNA leads to RNaseH-mediated degradation or inactivation of the target RNA and thus, limits the amount of available CD73 mRNA for protein expression.

The oligonucleotide of the present invention comprises the one or more, two or more, three or more or four or more modified nucleotide(s) at the 3′- and/or 5′-end of the oligonucleotide and/or at any position within the oligonucleotide, wherein modified nucleotides follow in a row of 1, 2, 3, 4, 5, or 6 modified nucleotides, or a modified nucleotide is combined with one or more, two or more, three or more or four or more unmodified nucleotides. The following Table 1 presents embodiments of oligonucleotides comprising modified nucleotides for example LNA which are indicated by (+) and phosphorothioate (PTO) indicated by (*). The oligonucleotides consisting of or comprising the sequences of Table 1 may comprise any other modified nucleotide and any other combination of modified and unmodified nucleotides. Oligonucleotides of Table 1 hybridize with human CD73 mRNA:

TABLE 1

| | | | | Position on pre-mRNA |
|---|---|---|---|---|
| SEQ | | Antisense | | (GRCh38.p13:6: |
| ID | | Sequence | Antisense Sequence 5'-3' | 85450083:85495784 |
| NO. | Name | 5′-3′ | with PTO (*) and LNA (+) | (SEQ ID NO. 2)) |
| 3 | A05046H | GCGAGTGCCGG | +G*+C*+G*A*G*T*G*C*C*G*G*C | 1 |
| | | CGAGT | *G*+A*+G*+T | |
| 4 | A05047H | AGCTGTGGCGC | +A*+G*+C*T*G*T*G*G*C*G*C*G | 41 |
| | | GTGAAC | *T*G*+A*+A*+C | |
| 5 | A05048H | GCTGGCGTTGA | +G*+C*+T*G*G*C*G*T*T*G*A*C | 206 |
| | | CGCACT | *G*C*+A*+C*+T | |
| 6 | A05049H | CCTGGTACTGG | +C*+C*+T*G*G*T*A*C*T*G*G*T* | 306 |
| | | TCGCCG | C*G*+C*+C*+G | |
| 7 | A05050H | CCGTGTGTCTCA | +C*+C*+G*T*G*T*G*T*C*T*C*A* | 39420 |
| | | GGTTG | G*G*+T*+T*+G | |
| 8 | A05051H | CACGCTATGCTC | +C*+A*+C*G*C*T*A*T*G*C*T*C* | 40516 |
| | | AAAGG | A*A*+A*+G*+G | |
| 9 | A05052H | TCATACACCACA | +T*+C*+A*T*A*C*A*C*C*A*C*A* | 41897 |
| | | TGGAT | T*G*+G*+A*+T | |
| 10 | A05053HM | GGCACTCGACA | +G*+G*+C*A*C*T*C*G*A*C*A*C | 41963 |
| | | CTTGGT | *T*T*+G*+G*+T | |
| 11 | A05054H | CTTATATACCTC | +C*+T*+T*A*T*A*T*A*C*C*T*C* | 42000 |
| | | GTCCA | G*T*+C*+C*+A | |
| 12 | A05055H | GTAGAAACCAC | +G*+T*+A*G*A*A*A*C*C*A*C*G | 43770 |
| | | GTTGAT | *T*T*+G*+A*+T | |
| 13 | A05056H | GCTGTATGGTC | +G*+C*+T*G*T*A*T*G*G*T*C*A* | 44386 |
| | | AAGTCA | A*G*+T*+C*+A | |
| 14 | A05057H | CTCGTGTCCTTT | +C*+T*+C*G*T*G*T*C*C*T*T*T* | 44714 |
| | | GACTG | G*A*+C*+T*+G | |
| 15 | A05058H | TAGAACCGAGG | +T*+A*+G*A*A*C*C*G*A*G*G*C | 45445 |
| | | CTATTA | *T*A*+T*+T*+A | |
| 16 | A05059H | GGACACATAGC | +G*+G*+A*C*A*C*A*T*A*G*C*T* | 48 |
| | | TGTGGCG | G*T*G*+G*+C*+G | |
| 17 | A05060H | CTGTGCACGTC | +C*+T*+G*T*G*C*A*C*G*T*C*G* | 156 |
| | | GTTGGTG | T*T*G*+G*+T*+G | |
| 18 | A05061H | TGGAGTCCTCG | +T*+G*+G*A*G*T*C*C*T*C*G*C* | 185 |
| | | CTGGTCT | T*G*G*+T*+C*+T | |
| 19 | A05062H | TTGACGCACTTG | +T*+T*+G*A*C*G*C*A*C*T*T*G* | 198 |
| | | CTGGAG | C*T*G*+G*+A*+G | |
| 20 | A05063H | ATGGCATCGTA | +A*+T*+G*G*C*A*T*C*G*T*A*G* | 378 |
| | | GCGCAGG | C*G*C*+A*+G*+G | |
| 21 | A05064H | GGAAATTTGGC | +G*+G*+A*A*A*T*T*T*G*G*C*C* | 17034 |
| | | CTCTTTG | T*C*T*+T*+T*+G | |
| 22 | A05065H | AAGTGTATCCAA | +A*+A*+G*T*G*T*A*T*C*C*A*A* | 17153 |
| | | CGATTC | C*G*A*+T*+T*+C | |
| 23 | A05066H | ATCTACTTCAGG | +A*+T*+C*T*A*C*T*T*C*A*G*G* | 21190 |
| | | TTGTAA | T*T*G*+T*+A*+A | |
| 24 | A05067H | TTTGTTCACATT | +T*+T*+T*G*T*T*C*A*C*A*T*T* | 21217 |
| | | TAGAGT | T*A*G*+A*+G*+T | |
| 25 | A05068H | CTTTCTGAGCGA | +C*+T*+T*T*C*T*G*A*G*C*G*A* | 21272 |
| | | TGAGTT | T*G*A*+G*+T*+T | |
| 26 | A05069H** | GTGGATTGCCT | +G*+T*+G*G*A*T*T*G*C*C*T*G* | 800* |
| | | GTGTAAA | T*G*T*+A*+A*+A | |
| 27 | A05070H | CTACAGGAACC | +C*+T*+A*C*A*G*G*A*A*C*C*T* | 35216 |
| | | TTCCGCC | T*C*C*+G*+C*+C | |
| 28 | A05071H | GCATAGGCCTG | +G*+C*+A*T*A*G*G*C*C*T*G*G | 35229 |
| | | GACTACA | *A*C*T*+A*+C*+A | |
| 29 | A05072H | TTCAGATAGCCT | +T*+T*+C*A*G*A*T*A*G*C*C*T* | 35256 |
| | | AGGTAT | A*G*G*+T*+A*+T | |
| 30 | A05073H | AACTCGATCTTC | +A*+A*+C*T*C*G*A*T*C*T*T*C* | 35265 |
| | | AGATAG | A*G*A*+T*+A*+G | |
| 31 | A05074H | CTCTTTCATCAA | +C*+T*+C*T*T*T*C*A*T*C*A*A* | 35276 |
| | | ACTCGA | A*C*T*+C*+G*+A | |
| 32 | A05075H** | TTATGCTTGGAT | +T*+T*+A*T*G*C*T*T*G*G*A*T* | 1001 |
| | | CTTCAG | C*T*T*+C*+A*+G | |
| 33 | A05076H | AGGAGCCATCC | +A*+G*+G*A*G*C*C*A*T*C*C*A | 37334 |
| | | AGATAGA | *G*A*T*+A*+G*+A | |
| 34 | A05077H | GGATACCACCT | +G*+G*+A*T*A*C*C*A*C*C*T*C* | 39473 |
| | | CCATTTA | C*A*T*+T*+T*+A | |
| 35 | A05078H** | AGGTAATTGTG | +A*+G*+G*T*A*A*T*T*G*T*G*C* | 1262 |
| | | CCATTGT | C*A*T*+T*+G*+T | |
| 36 | A05079H | GTGGAACCTTTT | +G*+T*+G*G*A*A*C*C*T*T*T*T* | 40487 |
| | | AACTGG | A*A*C*+T*+G*+G | |
| 37 | A05080H | AGTGGACTGGC | +A*+G*+T*G*G*A*C*T*G*G*C*C | 40536 |
| | | CGTAGCG | *G*T*A*+G*+C*+G | |
| 38 | A05081H | CGACACTTGGT | +C*+G*+A*C*A*C*T*T*G*G*T*G* | 41956 |
| | | GCAAAGA | C*A*A*+A*+G*+A | |
| 39 | A05082H | ATACCTCGTCCA | +A*+T*+A*C*C*T*C*G*T*C*C*A* | 41994 |
| | | TTTTGA | T*T*T*+T*+G*+A | |
| 40 | A05083H | CGACCTTCAACT | +C*+G*+A*C*C*T*T*C*A*A*C*T* | 43814 |
| | | GCTGGA | G*C*T*+G*+G*+A | |
| 41 | A05084H | AACTTGATCCGA | +A*+A*+C*T*T*G*A*T*C*C*G*A* | 43823 |
| | | CCTTCA | C*C*T*+T*+C*+A | |
| 42 | A05085H | CCTGTGGAAAA | +C*+C*+T*G*T*G*G*A*A*A*A*C* | 43832 |
| | | CTTGATC | T*T*G*+A*+T*+C | |
| 43 | A05086H | GGTCCTAAAAG | +G*+G*+T*C*C*T*A*A*A*A*G*G | 43979 |
| | | GCAGATT | *C*A*G*+A*+T*+T | |
| 44 | A05087H | ACGTAAACTTAA | +A*+C*+G*T*A*A*A*C*T*T*A*A* | 44148 |
| | | CATAGT | C*A*T*+A*+G*+T | |
| 45 | A05088H | TCAGCATTAGCT | +T*+C*+A*G*C*A*T*T*A*G*C*T* | 44394 |
| | | GTATGG | G*T*A*+T*+G*+G | |
| 46 | A05089H | GGATCTGTCAG | +G*+G*+A*T*C*T*G*T*C*A*G*C* | 44401 |
| | | CATTAGC | A*T*T*+A*+G*+C | |
| 47 | A05090H | GATTTGTCATCC | +G*+A*+T*T*T*G*T*C*A*T*C*C* | 44528 |
| | | ATGAGC | A*T*G*+A*+G*+C | |
| 48 | A05091H | ACTCATCAAAG | +A*+C*+T*C*A*T*C*A*A*A*G*G* | 44646 |
| | | GCACATG | C*A*C*+A*+T*+G | |
| 49 | A05092H | ATTATCTACTAC | +A*+T*+T*A*T*C*T*A*C*T*A*C* | 44671 |
| | | AGCTTG | A*G*C*+T*+T*+G | |
| 50 | A05093H | ATAACAGCTAAT | +A*+T*+A*A*C*A*G*C*T*A*A*T* | 44843 |
| | | GCCGTG | G*C*C*+G*+T*+G | |
| 51 | A05094H | GCTTATGTTAGA | +G*+C*+T*T*A*T*G*T*T*A*G*A* | 44926 |
| | | AGGTTC | A*G*G*+T*+T*+C | |
| 52 | A05095H | TCGAGAACTCT | +T*+C*+G*A*G*A*A*C*T*C*T*G* | 45028 |
| | | GGACATT | G*A*C*+A*+T*+T | |
| 53 | A05096H | TGGAGGCAGAG | +T*+G*+G*A*G*G*C*A*G*A*G*C | 45083 |
| | | CGACTTA | *G*A*C*+T*+T*+A | |
| 54 | A05097H | GGCATAGGTCA | +G*+G*+C*A*T*A*G*G*T*C*A*T* | 45200 |
| | | TTTCATC | T*T*C*+A*+T*+C | |
| 55 | A05098HI | CGAGAGTATGC | +C*+G*+A*G*A*G*T*A*T*G*C*T* | 19245 |
| | | TACCA | A*+C*+C*+A | |
| 56 | A05099HI | TGCGGCCGAGC | +T*+G*+C*G*G*C*C*G*A*G*C*C | 43104 |
| | | CATTG | *A*+T*+T*+G | |
| 57 | A05100HI | GAATCAATATGC | +G*+A*+A*T*C*A*A*T*A*T*G*C* | 2745 |
| | | GGTGA | G*G*+T*+G*+A | |
| 58 | A05101HI | GTAACAAACGA | +G*+T*+A*A*C*A*A*A*C*G*A*T* | 20230 |
| | | TAGCCT | A*G*+C*+C*+T | |
| 59 | A05102HI | TGGTTGCAAACT | +T*+G*+G*T*T*G*C*A*A*A*C*T* | 20892 |
| | | GTGAG | G*T*+G*+A*+G | |
| 60 | A05103HI | GTAGTCCGACA | +G*+T*+A*G*T*C*C*G*A*C*A*T* | 38153 |
| | | TAGGAG | A*G*+G*+A*+G | |
| 61 | A05104HI | TTAGATCTGCTA | +T*+T*+A*G*A*T*C*T*G*C*T*A* | 40152 |
| | | GCTTG | G*C*+T*+T*+G | |
| 62 | A05105HI | GAGCCATTGGT | +G*+A*+G*C*C*A*T*T*G*G*T*A* | 43096 |
| | | ATTTAA | T*T*+T*+A*+A | |
| 63 | A05106HI | CACACTCTGCCA | +C*+A*+C*A*C*T*C*T*G*C*C*A* | 462 |
| | | TCCGCT | T*C*C*+G*+C*+T | |
| 64 | A05107HI | GATCTGGTGTC | +G*+A*+T*C*T*G*G*T*G*T*C*C* | 875 |
| | | CATTCTT | A*T*T*+C*+T*+T | |
| 65 | A05108HI | TAGCTGTGGAA | +T*+A*+G*C*T*G*T*G*G*A*A*T* | 2002 |
| | | TACCAAT | A*C*C*+A*+A*+T | |
| 66 | A05109HI | GCCATCTAACCT | +G*+C*+C*A*T*C*T*A*A*C*C*T* | 2516 |
| | | TGCCAT | T*G*C*+C*+A*+T | |
| 67 | A05110HI | ATCAATATGCG | +A*+T*+C*A*A*T*A*T*G*C*G*G* | 2742 |
| | | GTGAGTG | T*G*A*+G*+T*+G | |
| 68 | A05111HI | GGCTCCTTTGAA | +G*+G*+C*T*C*C*T*T*T*G*A*A* | 2834 |
| | | CTAGGT | C*T*A*+G*+G*+T | |
| 69 | A05112HI | CTAGAAAGTGT | +C*+T*+A*G*A*A*A*G*T*G*T*A* | 2943 |
| | | ACACCTC | C*A*C*+C*+T*+C | |
| 70 | A05113HI | CCTACAATAAAG | +C*+C*+T*A*C*A*A*T*A*A*A*G* | 4279 |
| | | CTGGAT | C*T*G*+G*+A*+T | |
| 71 | A05114HI | AGAAGTGAATT | +A*+G*+A*A*G*T*G*A*A*T*T*G* | 4737 |
| | | GCATAGC | C*A*T*+A*+G*+C | |
| 72 | A05115HI | CAGAGGTAAGC | +C*+A*+G*A*G*G*T*A*A*G*C*T | 4791 |
| | | TGGTTCA | *G*G*T*+T*+C*+A | |
| 73 | A05116HI | ATGCCAAGCTG | +A*+T*+G*C*C*A*A*G*C*T*G*T* | 17246 |
| | | TGATTTA | G*A*T*+T*+T*+A | |
| 74 | A05117HI | CTGTTTAGCACT | +C*+T*+G*T*T*T*A*G*C*A*C*T* | 17473 |
| | | GGCTAT | G*G*C*+T*+A*+T | |
| 75 | A05118HI | GTTACAGCCTG | +G*+T*+T*A*C*A*G*C*C*T*G*G* | 18767 |
| | | GTAAAGG | T*A*A*+A*+G*+G | |
| 76 | A05119HI | ATGTCGAGAGT | +A*+T*+G*T*C*G*A*G*A*G*T*A* | 19247 |
| | | ATGCTAC | T*G*C*+T*+A*+C | |
| 77 | A05120HI | AGATCCAGACG | +A*+G*+A*T*C*C*A*G*A*C*G*T* | 20003 |
| | | TTCTTAC | T*C*T*+T*+A*+C | |
| 78 | A05121HI | GGAGGCTTCAG | +G*+G*+A*G*G*C*T*T*C*A*G*A | 20875 |
| | | ATATTGT | *T*A*T*+T*+G*+T | |
| 79 | A05122HI | AAGGTGGAACC | +A*+A*+G*G*T*G*G*A*A*C*C*A | 22017 |
| | | AGATTCA | *G*A*T*+T*+C*+A | |
| 80 | A05123HI | TGGAACTTTGA | +T*+G*+G*A*A*C*T*T*T*G*A*G* | 22507 |
| | | GCATGAT | C*A*T*+G*+A*+T | |
| 81 | A05124HI | CTTAAGTGAAG | +C*+T*+T*A*A*G*T*G*A*A*G*G* | 22666 |
| | | GCCAACT | C*C*A*+A*+C*+T | |
| 82 | A05125HI | AATCCACGAGC | +A*+A*+T*C*C*A*C*G*A*G*C*T* | 25130 |
| | | TTTGGAA | T*T*G*+G*+A*+A | |
| 83 | A05126HI | GACTCTAGGATT | +G*+A*+C*T*C*T*A*G*G*A*T*T* | 25479 |
| | | TAACTT | T*A*A*+C*+T*+T | |
| 84 | A05127HI | CCGCAATAGAC | +C*+C*+G*C*A*A*T*A*G*A*C*T* | 35793 |
| | | TCAGACA | C*A*G*+A*+C*+A | |
| 85 | A05128HI | ACGCTCATCTTG | +A*+C*+G*C*T*C*A*T*C*T*T*G* | 36358 |
| | | CCGCCG | C*C*G*+C*+C*+G | |
| 86 | A05129HI | CATCTGGCTACT | +C*+A*+T*C*T*G*G*C*T*A*C*T* | 36857 |
| | | GAGAGG | G*A*G*+A*+G*+G | |
| 87 | A05130HI | ATTAGTGGTGG | +A*+T*+T*A*G*T*G*G*T*G*G*C* | 36962 |
| | | CGGTAGG | G*G*T*+A*+G*+G | |
| 88 | A05131HI | GGTACTGAGTA | +G*+G*+T*A*C*T*G*A*G*T*A*T* | 37729 |
| | | TGAAGCT | G*A*A*+G*+C*+T | |
| 89 | A05132HI | GTAGCAGAGTT | +G*+T*+A*G*C*A*G*A*G*T*T*T* | 37876 |
| | | TGTGCAT | G*T*G*+C*+A*+T | |
| 90 | A05133HI | GTAGTCCGACA | +G*+T*+A*G*T*C*C*G*A*C*A*T* | 38152 |
| | | TAGGAGA | A*G*G*+A*+G*+A | |
| 91 | A05134HI | TTGGCATGAGC | +T*+T*+G*G*C*A*T*G*A*G*C*A* | 38288 |
| | | ATGATTG | T*G*A*+T*+T*+G | |
| 92 | A05135HI | GATAAGCACTG | +G*+A*+T*A*A*G*C*A*C*T*G*C* | 38415 |
| | | CCAACAG | C*A*A*+C*+A*+G | |
| 93 | A05136HI | AATGGTCTCTCG | +A*+A*+T*G*G*T*C*T*C*T*C*G* | 39141 |
| | | GTTGTA | G*T*T*+G*+T*+A | |
| 94 | A05137HI | CCAGTCCATGTC | +C*+C*+A*G*T*C*C*A*T*G*T*C* | 39211 |
| | | AAACTC | A*A*A*+C*+T*+C | |
| 95 | A05138HI | GATTTACACTAG | +G*+A*+T*T*T*A*C*A*C*T*A*G* | 39378 |
| | | TTACTC | T*T*A*+C*+T*+C | |
| 96 | A05139HI | TTTAATCCAGTG | +T*+T*+T*A*A*T*C*C*A*G*T*G* | 39898 |
| | | GTATGT | G*T*A*+T*+G*+T | |
| 97 | A05140HI | CTTAGATCTGCT | +C*+T*+T*A*G*A*T*C*T*G*C*T* | 40152 |
| | | AGCTTG | A*G*C*+T*+T*+G | |
| 98 | A05141HI | TATTAGAAACTA | +T*+A*+T*T*A*G*A*A*A*C*T*A* | 40854 |
| | | GACCTC | G*A*C*+C*+T*+C | |
| 99 | A05142HI | ATGCAGTGCTTT | +A*+T*+G*C*A*G*T*G*C*T*T*T* | 41453 |
| | | GCTAGA | G*C*T*+A*+G*+A | |
| 100 | A05143HI | CACAAGGCATA | +C*+A*+C*A*A*G*G*C*A*T*A*G | 41469 |
| | | GAGCTAT | *A*G*C*+T*+A*+T | |
| 101 | A05144HI | GTCAGTGGTCT | +G*+T*+C*A*G*T*G*G*T*C*T*G* | 42659 |
| | | GTATGCA | T*A*T*+G*+C*+A | |
| 102 | A05145HI | TTGTAAGCATGC | +T*+T*+G*T*A*A*G*C*A*T*G*C* | 42712 |
| | | TGGTCT | T*G*G*+T*+C*+T | |
| 103 | A05146HI | GAGCCATTGGT | +G*+A*+G*C*C*A*T*T*G*G*T*A* | 43095 |
| | | ATTTAAT | T*T*T*+A*+A*+T | |
| 104 | A05147HI | GATAAATGCTAA | +G*+A*+T*A*A*A*T*G*C*T*A*A* | 43311 |
| | | TTGCCT | T*T*G*+C*+C*+T | |
| 105 | A05148HI | TTGAACCACTCC | +T*+T*+G*A*A*C*C*A*C*T*C*C* | 43464 |
| | | AGAACA | A*G*A*+A*+C*+A | |
| 106 | A05149HI | GGTAGTCCTTTG | +G*+G*+T*A*G*T*C*C*T*T*T*G* | 43683 |
| | | TAATTA | T*A*A*+T*+T*+A | |
| 107 | A05150HI | ACCAATGCTTAA | +A*+C*+C*A*A*T*G*C*T*T*A*A* | 1991 |
| | | GAGAC | G*A*+G*+A*+C | |
| 108 | A05151HI | ATCAATATGCG | +A*+T*+C*A*A*T*A*T*G*C*G*G* | 2743 |
| | | GTGAGT | T*G*+A*+G*+T | |
| 109 | A05152HI | CTAGGAATCAAT | +C*+T*+A*G*G*A*A*T*C*A*A*T* | 2749 |
| | | ATGCG | A*T*+G*+C*+G | |
| 110 | A05153HI | TGGTAACAAAC | +T*+G*+G*T*A*A*C*A*A*A*C*G* | 20232 |
| | | GATAGC | A*T*+A*+G*+C | |
| 111 | A05154H | CGGTGAACCAG | +C*+G*+G*T*G*A*A*C*C*A*G*A | 324 |
| | | ATAGTG | *T*A*+G*+T*+G | |
| 112 | A05155H | GTGTATCCAAC | +G*+T*+G*T*A*T*C*C*A*A*C*G* | 17152 |
| | | GATTCC | A*T*+T*+C*+C | |
| 113 | A05156H | GCGATGAGTTT | +G*+C*+G*A*T*G*A*G*T*T*T*A* | 21265 |
| | | ATCCAT | T*C*+C*+A*+T | |
| 114 | A05157H | ACCTTATATACC | +A*+C*+C*T*T*A*T*A*T*A*C*C* | 42002 |
| | | TCGTC | T*C*+G*+T*+C | |
| 115 | Neg1 | | +C*+G*+T*T*T*A*G*G*C*T*A*T* | |
| | | | G*T*A*+C*+T*+T | |

List of human CD73-specific ASOs and Control. An “H” after the ASO ID indicates a human CD73-specific sequence that binds to an exonic region of the pre-mRNA, a “HM” after the ASO ID indicates a human/mouse cross-reactive CD73 sequence that binds to an exonic region of the pre-mRNA and a “HI” after the ASO ID indicates a human CD73-specific sequence that binds to an intronic region of the pre-mRNA for example of SEQ ID NO.2 (GRCh38.p13:6:85450083:85495784).
*refers to exon spanning oligonucleotides, position depicted in Table 1 indicates positions on mRNA of SEQ ID NO. 1 (RefSeq ID NM_002526.4) for exon spanning oligonucleotides.

The oligonucleotides of the present invention hybridize for example with mRNA and/or pre-mRNA of human CD73 of SEQ ID NO. 1 and/or SEQ ID NO.2. Such oligonucleotides are called CD73 antisense oligonucleotides. Oligonucleotides of the present invention, which are for example antisense oligonucleotides, are shown in Table 1. The present invention further refers to oligonucleotides such as antisense oligonucleotides having 80 to 99%, 85 to 98%, 90 to 95 or 93% sequence homology to an oligonucleotide of Table 1. Each nucleotide of the sequence can be modified, wherein ASOs of the present invention preferably comprise a core of 6 to 8 unmodified nucleotides. ASOs of the present invention comprise for example one or more modified nucleotides, e.g., 1, 2, 3, 4 or 5 nucleotides at the 5′- and/or 3′-end of the oligonucleotide, i.e., on the 5′- and/or 3′-side of the core. The 5′- and 3′-end are modified identically or differently. If the 5′- and 3′-ends are modified identically the nucleotides are modified at the same positions counted from the 5′- and 3′-end (in each case starting the counting with 1 from the end), respectively, having the same modification for example LNA-modification. If the 5′- and 3′-ends are modified differently the position of the modified nucleotide and/or the type of modification at the 5′- and 3′-ends differ; the type of nucleotide modification is the same (e.g., LNA) or different. Modified nucleotides such as LNA-modified nucleotides need not to follow in a row, but may be separated by one or more unmodified nucleotides. In the following some modification patterns at the 5′- and 3′-end of the ASOs of the present invention are described, wherein an unmodified nucleotide is indicated by “_” and the figure refers to the number of modified nucleotides such as LNA-modified nucleotides in a row. The modified nucleotide(s) is/are at any position of the 5′- and/or 3′-end of the ASO as shown in the following Table 2:

| LNA modification at the | LNA modification at the | |
|---|---|---|
| 5′-side of the core | 3′-side of the core | Abbreviation |
| 3 | 3 | 3 + 3 |
| 3 | 2 | 3 + 2 |
| 2 | 3 | 2 + 3 |
| 1_1 | 3 | 1_1 + 3 |
| 1_1 | 2 | 1_1 + 2 |
| 1_1 | 1_1 | 1_1 + 1_1 |
| 3 | 1_1 | 3 + 1_1 |
| 2 | 1_1 | 2 + 1_1 |
| 2 | 2 | 2 + 2 |
| 4 | 3 | 4 + 3 |
| 4 | 2 | 4 + 2 |
| 4 | 1_1 | 4 + 1_1 |
| 2_1 | 3 | 2_1 + 3 |
| 2_1 | 1_1 | 2_1 + 1_1 |
| 2_1 | 2 | 2_1 + 2 |
| 3 | 4 | 3 + 4 |
| 2 | 4 | 2 + 4 |
| 1_1 | 4 | 1_1 + 4 |
| 3 | 1_2 | 3 + 1_2 |
| 1_1 | 1_2 | 1_1 + 1_2 |
| 2 | 1_2 | 2 + 1_2 |

Typical modification patterns of each ASO of the present invention, comprising for example LNA-modified nucleotides, are shown for example in the following Table 3 which indicates specific positions of the LNA modifications at the 5′- and 3′-end of each ASO:

| Position of the | Position of the | |
|---|---|---|
| modification at the 5′-end | modification at the 3′-end | |
| (counted from the 5′-end | (counted from the 3′-end | |
| starting with 1) | starting with 1) | Abbreviation |
| nucleotides 1 to 5 | nucleotides 1 to 5 | 5 + 5 |
| nucleotides 1 to 4 | nucleotides 1 to 4 | 4 + 4 |
| nucleotides 1 to 3 | nucleotides 1 to 3 | 3 + 3 |
| nucleotides 1 and 2 | nucleotides 1 and 2 | 2 + 2 |
| nucleotide 1 | nucleotide 1 | 1 + 1 |
| nucleotides 1 to 5 | nucleotides 1 to 4 | 5 + 4 |
| nucleotides 1 to 4 | nucleotides 1 to 3 | 4 + 3 |
| nucleotides 1 to 3 | nucleotides 1 and 2 | 3 + 2 |
| nucleotides 1 and 2 | nucleotide 1 | 2 + 1 |
| nucleotide 1 | nucleotides 1 to 5 | 1 + 5 |
| nucleotides 1 to 5 | nucleotides 1 to 3 | 5 + 3 |
| nucleotides 1 to 4 | nucleotides 1 and 2 | 4 + 2 |
| nucleotides 1 to 3 | nucleotide 1 | 3 + 1 |
| nucleotides 1 and 2 | nucleotides 1 to 5 | 2 + 5 |
| nucleotide 1 | nucleotides 1 to 4 | 1 + 4 |
| nucleotides 1 to 5 | nucleotides 1 and 2 | 5 + 2 |
| nucleotides 1 to 4 | nucleotide 1 | 4 + 1 |
| nucleotides 1 to 3 | nucleotides 1 to 5 | 3 + 5 |
| nucleotides 1 and 2 | nucleotides 1 to 4 | 2 + 4 |
| nucleotide 1 | nucleotides 1 to 3 | 1 + 3 |
| nucleotides 1 to 5 | nucleotide 1 | 5 + 1 |
| nucleotides 1 to 4 | nucleotides 1 to 5 | 4 + 5 |
| nucleotides 1 to 3 | nucleotides 1 to 4 | 3 + 4 |
| nucleotides 1 and 2 | nucleotides 1 to 3 | 2 + 3 |
| nucleotide 1 | nucleotides 1 and 2 | 1 + 2 |
| nucleotides 1 and 2 | nucleotides 1 and 3 | 2 + 1_1 |
| nucleotides 1 and 3 | nucleotides 1 to 3 | 1_1 + 3 |
| nucleotides 1 and 3 | nucleotides 1 and 3 | 1_1 + 1_1 |
| nucleotides 1 to 3 | nucleotides 1 and 3 | 3 + 1_1 |
| nucleotides 1 to 4 | nucleotides 1 and 3 | 4 + 1_1 |
| nucleotides 1, 2 and 4 | nucleotides 1 to 3 | 2_1 + 3 |
| nucleotides 1, 2 and 4 | nucleotides 1 and 3 | 2_1 + 1_1 |
| nucleotides 1, 2 and 4 | nucleotides 1 and 2 | 2_1 + 2 |
| nucleotides 1 and 3 | nucleotides 1 to 4 | 1_1 + 4 |
| nucleotides 1 to 3 | nucleotides 1, 2 and 4 | 3 + 1_2 |
| nucleotides 1 and 3 | nucleotides 1, 2 and 4 | 1_1 + 1_2 |
| nucleotides 1 and 2 | nucleotides 1, 2 and 4 | 2 + 1_2 |

The oligonucleotides of the present invention hybridize with hybridizing active regions of SEQ ID NO.2. In the present invention surprisingly several hybridizing active regions were identified for example selected from position 1 to 400, position 401 to 800, position 801 to 1200, position 1601 to 2000, position 2001 to 2400, position 2401 to 2800, position 2801 to 3200, position 4001 to 4400, position 4401 to 4800, position 16801 to 17200, position 17201 to 17600, position 18401 to 18800, position 19201 to 19600, position 20001 to 20400, position 20801 to 21200, position 21201 to 21600, position 22001 to 22400, position 22401 to 22800, position 24801 to 25200, position 25201 to 25600, position 35201 to 35600, position 35601 to 3600, position 36001 to 36400, position 36801 to 37200, position 37201 to 37600, position 37601 to 38000, position 38001 to 38400, position 38401 to 38800, position 38801 to 39200, position 39201 to 39600, position 39601 to 40000, position 40001 to 40400, position 40401 to 40800, position 40801 to 41200, position 41201 to 41600, position 41601 to 42000, position 42001 to 42400, position 42401 to 42800, position 42801 to 43200, position 43201 to 43600, position 43601 to 44000, position 44001 to 44400, position 44401 to 44800, position 44801 to 45200, position 45201 to 45600, position 45201 to 45701, or a combination thereof (including the terminal figures of the ranges) of CD73 pre-mRNA for example of SEQ ID NO.2. These regions and the oligonucleotides hybridizing in the different regions are shown in the following Table 4:

| Region of | First position on | |
|---|---|---|
| SEQ ID NO.2 | SEQ ID NO.2 | SEQ ID NO. |
| 1-400 | | |
| A05046H | 1 | 3 |
| A05047H | 41 | 4 |
| A05048H | 206 | 5 |
| A05049H | 306 | 6 |
| A05059H | 48 | 16 |
| A05060H | 156 | 17 |
| A05061H | 185 | 18 |
| A05062H | 198 | 19 |
| A05063H | 378 | 20 |
| A05154H | 324 | 11 |
| 401-800 | | |
| A05106HI | 462 | 63 |
| 801-1200 | | |
| A05107HI | 875 | 64 |
| 1601-2000 | | |
| A05150HI | 1991 | 107 |
| 2001-2400 | | |
| A05108HI | 2002 | 65 |
| 2401-2800 | | |
| A05100HI | 2745 | 57 |
| A05109HI | 2516 | 66 |
| A05110HI | 2742 | 67 |
| A05151HI | 2743 | 108 |
| A05152HI | 2749 | 109 |
| 2801-3200 | | |
| A05111HI | 2834 | 68 |
| A05112HI | 2943 | 69 |
| 4001-4400 | | |
| A05113HI | 4279 | 70 |
| 4401-4800 | | |
| A05114HI | 4737 | 71 |
| A05115HI | 4791 | 72 |
| 16801-17200 | | |
| A05064H | 17034 | 21 |
| A05065H | 17153 | 22 |
| A05155H | 17152 | 112 |
| 17201-17600 | | |
| A05116HI | 17246 | 73 |
| A05117HI | 17473 | 74 |
| 18401-18800 | | |
| A05118HI | 18767 | 75 |
| 19201-19600 | | |
| A05098HI | 19245 | 55 |
| A05119HI | 19247 | 76 |
| 20001-20400 | | |
| A05101HI | 20230 | 58 |
| A05120HI | 20003 | 77 |
| A05153HI | 20232 | 110 |
| 20801-21200 | | |
| A05066H | 21190 | 23 |
| A05102HI | 20892 | 59 |
| A05121HI | 20875 | 78 |
| 21201-21600 | | |
| A05067H | 21217 | 24 |
| A05068H | 21272 | 25 |
| A05156H | 21265 | 113 |
| 22001-22400 | | |
| A05122HI | 22017 | 79 |
| 22401-22800 | | |
| A05123HI | 22507 | 80 |
| A05124HI | 22666 | 81 |
| 24801-25200 | | |
| A05125HI | 25130 | 82 |
| 25201-25600 | | |
| A05126HI | 25479 | 83 |
| 35201-35600 | | |
| A05070H | 35216 | 27 |
| A05071H | 35229 | 28 |
| A05072H | 35256 | 29 |
| A05073H | 35265 | 30 |
| A05074H | 35276 | 31 |
| 35601-36000 | | |
| A05127HI | 35793 | 84 |
| 36001-36400 | | |
| A05128HI | 36358 | 85 |
| 36801-37200 | | |
| A05129HI | 36857 | 86 |
| A05130HI | 36962 | 87 |
| 37201-37600 | | |
| A05076H | 37334 | 33 |
| 37601-38000 | | |
| A05131HI | 37729 | 88 |
| A05132HI | 37876 | 89 |
| 38001-38400 | | |
| A05103HI | 38153 | 60 |
| A05133HI | 38152 | 90 |
| A05134HI | 38288 | 91 |
| 38401-38800 | | |
| A05135HI | 38415 | 92 |
| 38801-39200 | | |
| A05136HI | 39141 | 93 |
| 39201-39600 | | |
| A05050H | 39420 | 7 |
| A05077H | 39473 | 34 |
| A05137HI | 39211 | 94 |
| A05138HI | 39378 | 95 |
| 39601-40000 | | |
| A05139HI | 39898 | 96 |
| 40001-40400 | | |
| A05104HI | 40152 | 61 |
| A05140HI | 40152 | 97 |
| 40401-40800 | | |
| A05051H | 40516 | 8 |
| A05079H | 40487 | 36 |
| A05080H | 40536 | 37 |
| 40801-41200 | | |
| A05141HI | 40854 | 98 |
| 41201-41600 | | |
| A05142HI | 41453 | 99 |
| A05143HI | 41469 | 100 |
| 41601-42000 | | |
| A05052H | 41897 | 9 |
| A05053HM | 41963 | 10 |
| A05054H | 42000 | 11 |
| A05081H | 41956 | 38 |
| A05082H | 41994 | 39 |
| 42001-42400 | | |
| A05157H | 42002 | 114 |
| 42401-42800 | | |
| A05144HI | 42659 | 101 |
| A05145HI | 42712 | 102 |
| 42801-43200 | | |
| A05099HI | 43104 | 56 |
| A05105HI | 43096 | 62 |
| A05146HI | 43095 | 103 |
| 43201-43600 | | |
| A05147HI | 43311 | 104 |
| A05148HI | 43464 | 105 |
| 43601-44000 | | |
| A05055H | 43770 | 12 |
| A05083H | 43814 | 40 |
| A05084H | 43823 | 41 |
| A05085H | 43832 | 42 |
| A05086H | 43979 | 43 |
| A05149HI | 43683 | 106 |
| 44001-44400 | | |
| A05056H | 44386 | 13 |
| A05087H | 44148 | 44 |
| A05088H | 44394 | 45 |
| 44401-44800 | | |
| A05057H | 44714 | 14 |
| A05089H | 44401 | 46 |
| A05090H | 44528 | 47 |
| A05091H | 44646 | 48 |
| A05092H | 44671 | 49 |
| 44801-45200 | | |
| A05093H | 44843 | 50 |
| A05094H | 44926 | 51 |
| A05095H | 45028 | 52 |
| A05096H | 45083 | 53 |
| A05097H | 45200 | 54 |
| 45201-45701 | | |
| A05058H | 45445 | 15 |

Table 4 shows some hybridizing active regions and antisense oligonucleotides hybridizing in these regions.

In some embodiments, the oligonucleotide of the present invention reduces the amount of CD73 mRNA and/or the CD73 protein expression for example about 30%-100%, 35%-99%, 40%-98%, 45%-97%, 50%-96%, 55%-95%, 60%-90%, 65%-85%, 70%-80% or at least about 30%, 35%, 40%, 45%, 50%, 55%, 60%, 65%, 70%, 75%, 80%, 85%, 90%, 92%, 94%, 95%, 96%, 97%, 98%, 99% or 100%. The reduction of the amount of the CD73 mRNA and/or CD73 protein expression is determined by the comparison of the amount of the CD73 mRNA and/or CD73 protein expression in a sample treated with an oligonucleotide of the present invention and a corresponding untreated control. The untreated control is for example CD73, CD73 mRNA, CD73 pre-mRNA expression or a combination thereof in a subject before an oligonucleotide of the present invention is administered or an untreated sample such as a cell, blood, urine, saliva etc. The untreated sample is for example taken from a subject before an oligonucleotide of the present invention is administered.

The oligonucleotides of the present invention are immunosuppression-reverting oligonucleotides which revert immunosuppression for example in a cell, tissue, organ, or a subject. The oligonucleotide of the present invention reduces the amount of CD73 mRNA and/or the expression of CD73 protein expression at a nanomolar or micromolar concentration for example at a concentration of 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 950 nM, or 1, 10 or 100 μM.

The oligonucleotide of the present invention is for example used in a concentration of 1, 3, 5, 9, 10, 15, 27, 30, 40, 50, 75, 82, 100, 250, 300, 500, or 740 nM, or 1, 2.2, 3, 5, 6.6 or P M.

The present invention also refers to a pharmaceutical composition comprising an oligonucleotide of the present invention and a pharmaceutically acceptable carrier, excipient and/or dilutant. Optionally, the pharmaceutical composition further comprises a chemotherapeutic, another oligonucleotide which is different from the present invention, an antibody and/or a small molecule.

The oligonucleotide or the pharmaceutical composition of the present invention is for example for use in a method of preventing and/or treating a disorder. The use of the oligonucleotide or the pharmaceutical composition of the present invention in a method of preventing and/or treating a disorder is for example combined with radiotherapy. The radiotherapy may be further combined with a chemotherapy (e.g., platinum, gemcitabine). The disorder is for example characterized by a CD73 mRNA and/or protein imbalance, i.e., the CD73 mRNA and/or protein level is increased in comparison to the level in a normal, healthy cell, tissue, organ or subject. The CD73 level is for example increased by an increased amount of CD73 mRNA and/or CD73 protein expression. The CD73 mRNA and protein level, respectively, can be measured by any standard method such as immunohistochemistry, western blot, quantitative real time PCR or QuantiGene assay known to a person skilled in the art.

An oligonucleotide or a pharmaceutical composition of the present invention is administered locally or systemically for example orally, sublingually, nasally, subcutaneously, intravenously, intraperitoneally, intramuscularly, intratumoral, intrathecal, transdermal and/or rectal. Alternatively or in combination ex vivo treated immune cells are administered. The oligonucleotide is administered alone or in combination with another oligonucleotide of the present invention and optionally in combination with another compound such as another oligonucleotide different from the present invention, an antibody, a small molecule and/or a chemotherapeutic (e.g., platinum, gemcitabine). In some embodiments, the other oligonucleotide (i.e., different from the present invention), the antibody, and/or the small molecule are effective in preventing and/or treating an autoimmune disorder for example autoimmune arthritis or gastrointestinal autoimmune diseases such as inflammatory bowel disease (IBD) or colitis, an immune disorder for example an immune exhaustion due to chronic viral infections such as HIV infection, a cardiovascular disorder, an inflammatory disorder for example a chronic airway inflammation, a bacterial, viral and/or fungal infection for example sepsis or a Mycobacterium bovis infection, a liver disorder, a chronic kidney disorder, a psychiatric disorder (e.g., schizophrenia, bipolar disorders, Alzheimer's disease) and/or cancer.

An oligonucleotide or a pharmaceutical composition of the present invention is used for example in a method of preventing and/or treating a solid tumor or a hematologic tumor. Examples of cancers preventable and/or treatable by use of the oligonucleotide or pharmaceutical composition of the present invention are breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomater tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythemia vera, adenocarcinoma, anaplastic astrocytoma, glioblastoma multiforme, leukemia, or epidermoid carcinoma.

Further, two or more oligonucleotides of the present invention are for example administered together, at the same time point, e.g., in a pharmaceutical composition or separately, or on staggered intervals. Alternatively or in addition, one or more oligonucleotides of the present invention are administered together with another compound such as another oligonucleotide (i.e., different from the present invention), an antibody, a small molecule and/or a chemotherapeutic, at the same time point for example in a pharmaceutical composition or separately, or on staggered intervals. In some of these combinations, the oligonucleotide of the present invention reduces for example the amount of CD73 mRNA and/or CD73 protein expression and the other oligonucleotide (i.e., different from the present invention), the antibody and/or small molecule inhibits (antagonist) or stimulates (agonist) the same and/or another immune suppressive factor and/or an immune stimulatory factor. The immune suppressive factor is for example selected from the group consisting of IDO1, IDO2, CTLA-4, PD-1, PD-L1, LAG-3, VISTA, A2AR, CD39, CD73, STAT3, TDO2, TIM-3, TIGIT, TGF-beta, BTLA, MICA, NKG2A, KIR, CD160, Chop, Xbp1 and a combination thereof. The immune stimulatory factor is for example selected from the group consisting of 4-1BB, Ox40, KIR, GITR, CD27, 2B4 and a combination thereof.

The immune suppressive factor is a factor whose expression and/or activity is for example increased in a cell, tissue, organ or subject. The immune stimulatory factor is a factor whose level is increased or decreased in a cell, tissue, organ or subject depending on the cell, tissue, organ or subject and its individual conditions.

An antibody in combination with the oligonucleotide or the pharmaceutical composition of the present invention is for example an anti-PD-1 antibody, an anti-PD-L1 antibody, or a bispecific antibody. A small molecule in combination with the oligonucleotide or the pharmaceutical composition of the present invention is for example AB680 or AMPCP (adenosine 5′-(α,β-methylene)diphosphate; e.g., Structure 20, 2161-2173, Dec. 5, 2012), which acts as an ADP analog and is therefore an competitive inhibitor of CD73 activity.

A subject of the present invention is for example a mammalian such as a human, dog, cat horse, cow, pig, a bird or a fish.

### EXAMPLES

The following examples illustrate different embodiments of the present invention, but the invention is not limited to these examples. The following experiments are performed on cells endogenously expressing CD73, i.e., the cells do not represent an artificial system comprising transfected reporter constructs. Such artificial systems generally show a higher degree of inhibition and lower IC5a values than endogenous systems which are closer to therapeutically relevant in vivo systems. Further, in the following experiments no transfecting agent is used, i.e., gymnotic delivery is performed. Transfecting agents are known to increase the activity of an oligonucleotide which influences the IC5a value (see for example Zhang et al., Gene Therapy, 2011, 18, 326-333; Stanton et al., Nucleic Acid Therapeutics, Vol. 22, No. 5, 2012). Since artificial systems using a transfecting agent are hard or impossible to translate into therapeutic approaches and no transfection formulation has been approved so far for oligonucleotides, the following experiments are performed without any transfecting agent.

### Example 1: Design of Human CD73-Specific Antisense Oligonucleotides (ASOs)

For the design of ASOs with specificity for exonic regions within the human CD73 gene the CD73 mRNA of SEQ ID NO.1 (RefSeq ID NM_002526.4) was used. For ASOs with specificity for intronic regions within the human CD73 gene the CD73 pre-mRNA of SEQ ID NO.2 (GRCh38.p13:6:85450083:85495784) was used. An “H” after the ASO ID indicates a human CD73-specific sequence that binds to an exonic region of the pre-mRNA, a “HM” after the ASO ID indicates a human/mouse cross-reactive CD73 sequence that binds to an exonic region of the pre-mRNA and a “HI” after the ASO ID indicates a human CD73-specific sequence that binds to an intronic region of the pre-mRNA. 16, 17 and 18 mers were designed according to in house criteria, neg1 was used as control oligonucleotide in all experiments. All the oligonucleotides and their sequences are shown in Table 1.

### Example 2: Target Knockdown Efficacy Screen of Human CD73-Specific ASOs

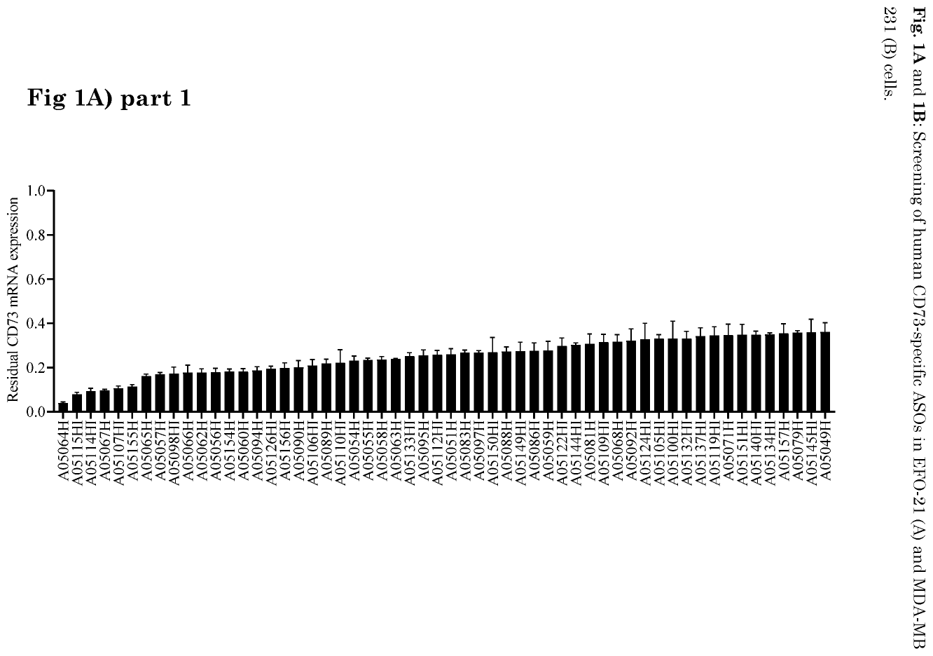

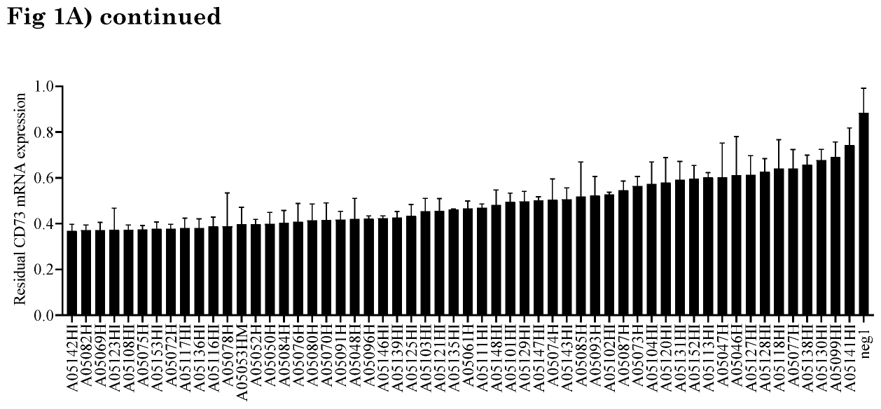

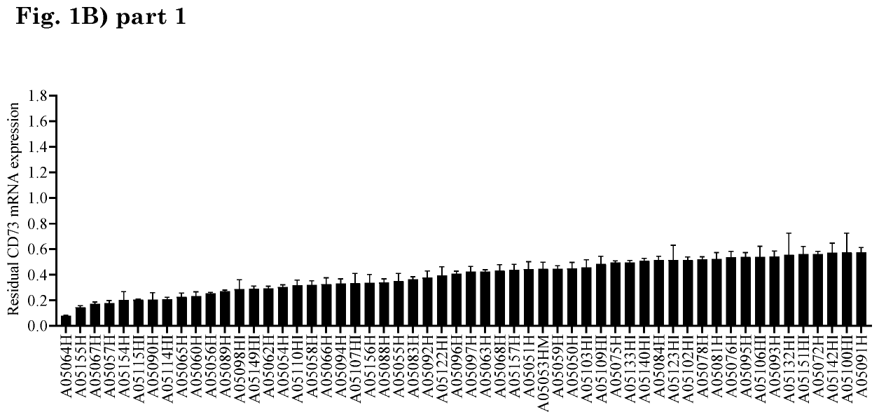

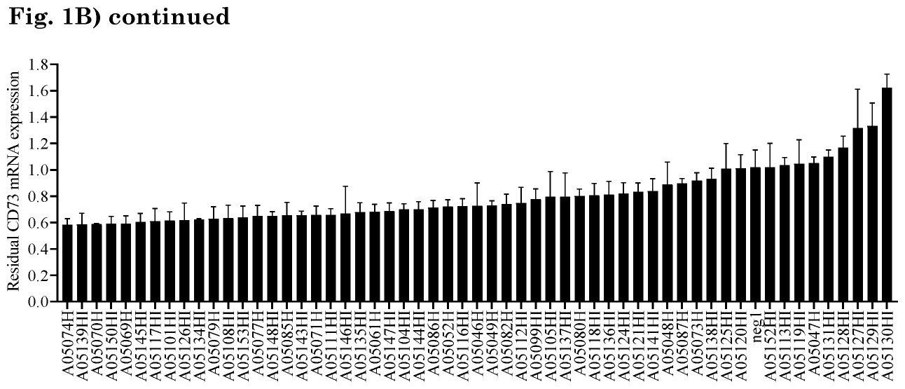

In order to investigate the knockdown efficacy of the in silico designed CD73-specific ASOs, two efficacy screening rounds in human cell lines were performed. Therefore, cells were treated with the respective ASO at a concentration of 5 μM for three days without the addition of a transfection reagent. Cells were lyzed after the three days treatment period, CD73 and HPRT1 mRNA expression was analyzed using the QuantiGene Singleplex assay (ThermoFisher) and the CD73 expression values were normalized to HPRT1 values. The results are shown in FIG. 1 as well as Tables 3 and 4.

As depicted in FIG. 1 A) and Table 5, treatment of EFO-21 cells with 36 of the 112 tested ASOs (34%) resulted in a target inhibition of >70% (represented by a residual CD73 mRNA expression of <0.3 as compared to mock treated cells). Knockdown efficacy of CD73-specific ASOs was furthermore tested in MDA-MB231 cells. As shown in FIG. 1B) and Table 6, treatment with 39 of the 112 tested ASOs (37%) resulted in a target inhibition of >50% (represented by a residual CD73 mRNA expression of <0.5 as compared to mock treated cells). The control oligonucleotide neg1 did not result in an inhibition of CD73 expression in both cell lines.

TABLE 5
List of the mean CD73 mRNA expression values in ASO-
treated EFO-21 cells compared to mock treated cells.
Expression values are normalized to HPRT1.

| | Residual CD73 mRNA |
|---|---|
| | expression (compared to |
| ASO | mock treated cells; set as 1) |
| A05064H | 0.04 |
| A05115HI | 0.08 |
| A05114HI | 0.09 |
| A05067H | 0.10 |
| A05107HI | 0.11 |
| A05155H | 0.11 |
| A05065H | 0.16 |
| A05057H | 0.17 |
| A05098HI | 0.17 |
| A05066H | 0.18 |
| A05062H | 0.18 |
| A05056H | 0.18 |
| A05154H | 0.18 |
| A05060H | 0.18 |
| A05094H | 0.19 |
| A05126HI | 0.19 |
| A05156H | 0.20 |
| A05090H | 0.20 |
| A05106HI | 0.21 |
| A05089H | 0.22 |
| A05110HI | 0.22 |
| A05054H | 0.23 |
| A05055H | 0.23 |
| A05058H | 0.24 |
| A05063H | 0.24 |
| A05133HI | 0.25 |
| A05095H | 0.25 |
| A05112HI | 0.26 |
| A05051H | 0.26 |
| A05083H | 0.27 |
| A05097H | 0.27 |
| A05150HI | 0.27 |
| A05088H | 0.27 |
| A05149HI | 0.27 |
| A05086H | 0.27 |
| A05059H | 0.28 |
| A05122HI | 0.30 |
| A05144HI | 0.30 |
| A05081H | 0.31 |
| A05109HI | 0.31 |
| A05068H | 0.32 |
| A05092H | 0.32 |
| A05124HI | 0.33 |
| A05105HI | 0.33 |
| A05100HI | 0.33 |
| A05132HI | 0.33 |
| A05137HI | 0.34 |
| A05119HI | 0.34 |
| A05071H | 0.35 |
| A05151HI | 0.35 |
| A05140HI | 0.35 |
| A05134HI | 0.35 |
| A05157H | 0.35 |
| A05079H | 0.36 |
| A05145HI | 0.36 |
| A05049H | 0.36 |
| A05142HI | 0.37 |
| A05082H | 0.37 |
| A05069H | 0.37 |
| A05123HI | 0.37 |
| A05108HI | 0.37 |
| A05075H | 0.37 |
| A05153HI | 0.38 |
| A05072H | 0.38 |
| A05117HI | 0.38 |
| A05136HI | 0.38 |
| A05116HI | 0.39 |
| A05078H | 0.39 |
| A05053HM | 0.40 |
| A05052H | 0.40 |
| A05050H | 0.40 |
| A05084H | 0.40 |
| A05076H | 0.41 |
| A05080H | 0.41 |
| A05070H | 0.42 |
| A05091H | 0.42 |
| A05048H | 0.42 |
| A05096H | 0.42 |
| A05146HI | 0.42 |
| A05139HI | 0.43 |
| A05125HI | 0.43 |
| A05103HI | 0.45 |
| A05121HI | 0.46 |
| A05135HI | 0.46 |
| A05061H | 0.47 |
| A05111HI | 0.47 |
| A05148HI | 0.48 |
| A05101HI | 0.49 |
| A05129HI | 0.50 |
| A05147HI | 0.50 |
| A05074H | 0.50 |
| A05143HI | 0.51 |
| A05085H | 0.52 |
| A05093H | 0.52 |
| A05102HI | 0.53 |
| A05087H | 0.55 |
| A05073H | 0.56 |
| A05104HI | 0.57 |
| A05120HI | 0.58 |
| A05131HI | 0.59 |
| A05152HI | 0.60 |
| A05113HI | 0.60 |
| A05047H | 0.60 |
| A05046H | 0.61 |
| A05127HI | 0.61 |
| A05128HI | 0.63 |
| A05118HI | 0.64 |
| A05077H | 0.64 |
| A05138HI | 0.66 |
| A05130HI | 0.68 |
| A05099HI | 0.69 |
| A05141HI | 0.74 |
| neg1 | 0.88 |

TABLE 6
List of the mean CD73 mRNA expression values in ASO-
treated MDA-MB 231 cells compared to mock treated
cells. Expression values are normalized to HPRT1.

| | Residual CD73 mRNA |
|---|---|
| | expression (compared to |
| ASO | mock treated cells; set as 1) |
| A05064H | 0.08 |
| A05155H | 0.15 |
| A05067H | 0.17 |
| A05057H | 0.18 |
| A05154H | 0.20 |
| A05115HI | 0.20 |
| A05090H | 0.20 |
| A05114HI | 0.21 |
| A05065H | 0.23 |
| A05060H | 0.23 |
| A05056H | 0.25 |
| A05089H | 0.27 |
| A05098HI | 0.29 |
| A05149HI | 0.29 |
| A05062H | 0.29 |
| A05054H | 0.30 |
| A05110HI | 0.32 |
| A05058H | 0.32 |
| A05066H | 0.33 |
| A05094H | 0.33 |
| A05107HI | 0.33 |
| A05156H | 0.34 |
| A05088H | 0.34 |
| A05055H | 0.35 |
| A05083H | 0.36 |
| A05092H | 0.38 |
| A05122HI | 0.39 |
| A05096H | 0.41 |
| A05097H | 0.42 |
| A05063H | 0.42 |
| A05068H | 0.43 |
| A05157H | 0.44 |
| A05051H | 0.44 |
| A05053HM | 0.44 |
| A05059H | 0.45 |
| A05050H | 0.45 |
| A05103HI | 0.46 |
| A05109HI | 0.48 |
| A05075H | 0.49 |
| A05133HI | 0.50 |
| A05140HI | 0.51 |
| A05084H | 0.51 |
| A05123HI | 0.51 |
| A05102HI | 0.52 |
| A05078H | 0.52 |
| A05081H | 0.52 |
| A05076H | 0.54 |
| A05095H | 0.54 |
| A05106HI | 0.54 |
| A05093H | 0.54 |
| A05132HI | 0.56 |
| A05151HI | 0.56 |
| A05072H | 0.56 |
| A05142HI | 0.57 |
| A05100HI | 0.57 |
| A05091H | 0.57 |
| A05074H | 0.58 |
| A05139HI | 0.59 |
| A05070H | 0.59 |
| A05150HI | 0.59 |
| A05069H | 0.59 |
| A05145HI | 0.61 |
| A05117HI | 0.61 |
| A05101HI | 0.61 |
| A05126HI | 0.62 |
| A05134HI | 0.62 |
| A05079H | 0.63 |
| A05108HI | 0.63 |
| A05153HI | 0.64 |
| A05077H | 0.65 |
| A05148HI | 0.65 |
| A05085H | 0.65 |
| A05143HI | 0.66 |
| A05071H | 0.66 |
| A05111HI | 0.66 |
| A05146HI | 0.67 |
| A05135HI | 0.68 |
| A05061H | 0.68 |
| A05147HI | 0.69 |
| A05104HI | 0.70 |
| A05144HI | 0.70 |
| A05086H | 0.71 |
| A05052H | 0.72 |
| A05116HI | 0.72 |
| A05046H | 0.73 |
| A05049H | 0.73 |
| A05082H | 0.74 |
| A05112HI | 0.75 |
| A05099HI | 0.78 |
| A05105HI | 0.79 |
| A05137HI | 0.80 |
| A05080H | 0.80 |
| A05118HI | 0.81 |
| A05136HI | 0.81 |
| A05124HI | 0.82 |
| A05121HI | 0.83 |
| A05141HI | 0.84 |
| A05048H | 0.89 |
| A05087H | 0.90 |
| A05073H | 0.92 |
| A05138HI | 0.93 |
| A05125HI | 1.01 |
| A05120HI | 1.01 |
| neg1 | 1.02 |
| A05152HI | 1.02 |
| A05113HI | 1.04 |
| A05119HI | 1.04 |
| A05047H | 1.05 |
| A05131HI | 1.10 |
| A05128HI | 1.17 |
| A05127HI | 1.32 |
| A05129HI | 1.33 |
| A05130HI | 1.62 |

### Example 3: Investigation of the Dose-Dependent Target Knockdown by Selected Human CD73-Specific ASOs

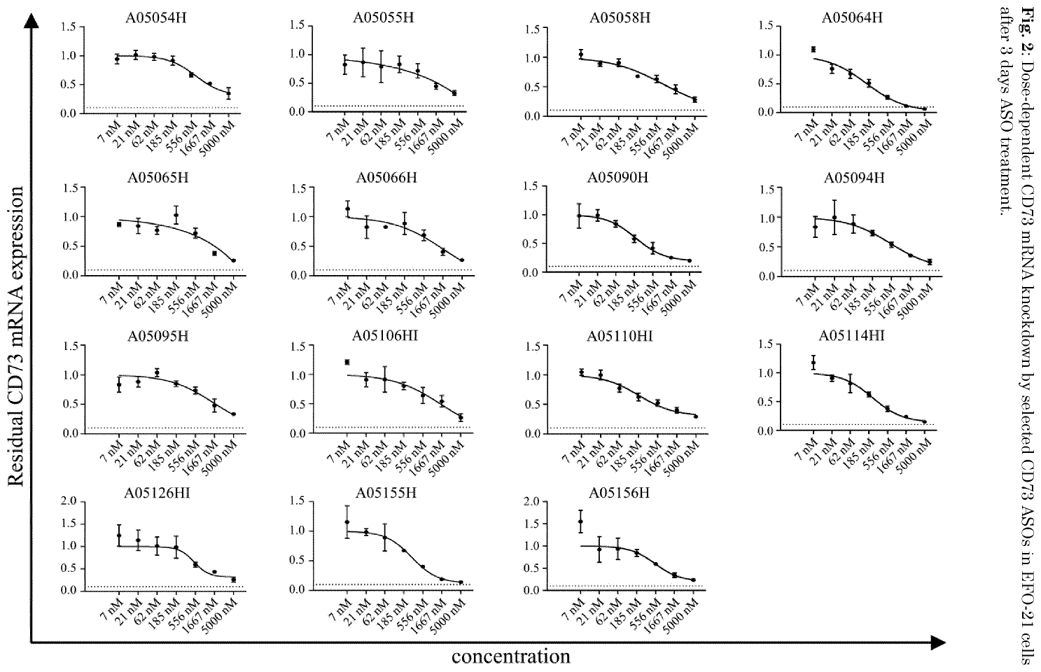

The dose-dependent knockdown of CD73 mRNA expression by CD73-specific ASOs in EFO-21 cells was investigated on mRNA level and the respective IC50 values were calculated. Therefore, EFO-21 cells were treated for three days with the respective ASO at the following concentrations: 5000 nM, 1667 nM, 556 nM, 185 nM, 62 nM, 21 nM, and 7 nM. After the treatment period, cells were lyzed, CD73 and HPRT1 mRNA expression was analyzed using the QuantiGene Singleplex assay (ThermoFisher) and the CD73 expression values were normalized to HPRT1 values (FIG. 2 and Table 7). A dose-dependent knockdown of CD73 mRNA was observed after treatment with all tested CD73-specific ASOs (FIG. 2) with IC5a values between 160.4 nM (A05064H; SEQ ID NO.21) and 1718 nM (A05066H; SEQ ID NO.23) (Table 7).

TABLE 7
Dose-dependent inhibition of CD73 mRNA expression in EFO-21 cells by selected
CD73 ASOs and respective IC₅₀ values after 3 days ASO treatment.

| | | Inhibition (%) | | | | | | |
|---|---|---|---|---|---|---|---|---|
| ASO | IC₅₀ (nM) | 7 nM | 21 nM | 62 nM | 185 nM | 556 nM | 1667 nM | 5000 nM |
| A05054H | 718.2 | 5.84 | −1.35 | 2.02 | 8.49 | 33.02 | 48.18 | 65.35 |
| A05055H | ambiguous | 17.61 | 13.34 | 21.04 | 16.75 | 28.17 | 55.40 | 67.35 |
| A05058H | 808.9 | −4.57 | 11.16 | 9.67 | 32.62 | 37.09 | 54.46 | 72.02 |
| A05064H | 160.4 | −10.04 | 23.68 | 32.50 | 48.63 | 73.41 | 88.45 | 93.55 |
| A05065H | ambiguous | 13.41 | 15.99 | 23.01 | −2.53 | 27.93 | 62.35 | 74.13 |
| A05066H | 1718 | −13.17 | 17.43 | 17.54 | 11.63 | 31.44 | 59.64 | 73.39 |
| A05090H | 202.2 | 1.91 | 1.03 | 16.10 | 41.90 | 58.20 | 74.99 | 80.05 |
| A05094H | 512.8 | 16.67 | 0.39 | 11.56 | 26.51 | 46.01 | 64.39 | 75.27 |
| A05095H | 1550 | 16.86 | 11.87 | −3.58 | 15.01 | 26.61 | 51.90 | 66.30 |
| A05106HI | 1571 | −20.92 | 9.22 | 8.77 | 19.96 | 36.08 | 45.95 | 73.69 |
| A05110HI | 189 | −4.31 | 0.48 | 22.87 | 37.55 | 47.58 | 60.01 | 70.92 |
| A05114HI | 230.1 | −17.88 | 9.13 | 18.60 | 37.60 | 62.45 | 76.26 | 85.34 |
| A05126HI | 513.4 | −24.92 | −14.28 | −1.24 | 1.35 | 40.23 | 56.52 | 73.89 |
| A05155H | 292.9 | −15.39 | 1.41 | 10.69 | 32.46 | 59.78 | 81.07 | 85.99 |
| A05156H | 539.5 | −55.01 | 7.68 | 6.33 | 15.54 | 40.18 | 65.53 | 76.43 |

### Example 4: Investigation of Potential Off-Target Binding Sites

Two different databases were screened in silico to test off-target effects of oligonucleotides of the present invention. These databases were RefSecRNA comprising sequences of spliced RNA and ENSEMBL comprising sequences of non-spliced RNA. The oligonucleotides shown in Tables 8 and 9 have no potential off-target binding site with zero mismatches, i.e., 100% sequence complementarity (0 mm) to an off-target sequence and/or one mismatch, i.e., ((n−1)/n*100) % sequence complementarity (1 mm) to an off-target sequence. The number of potential off-target sites of an oligonucleotide of the present invention having two mismatches, i.e., ((n−2)/n*100) % sequence complementarity (2 mm) is limited to max. 22 (see Tables 8 and 9, RefSeq (Gene Ids), 2 mm).

TABLE 8
Number of genes, besides the target gene, that show a sequence
complementarity with the respective CD73 exon-binding antisense
oligonucleotide allowing 0, 1 or 2 mismatches.

| | RefSeq (Gene Ids) | | | ENSEMBL | | |
|---|---|---|---|---|---|---|
| ASO | 0 mm | 1 mm | 2 mm | 0 mm | 1 mm | 2 mm |
| A05046H | 0 | 0 | 8 | 0 | 0 | 33 |
| A05047H | 0 | 0 | 4 | 0 | 0 | 15 |
| A05048H | 0 | 0 | 7 | 0 | 0 | 18 |
| A05049H | 0 | 0 | 10 | 0 | 0 | 15 |
| A05050H | 0 | 0 | 9 | 0 | 0 | 62 |
| A05051H | 0 | 0 | 4 | 0 | 0 | 49 |
| A05052H | 0 | 0 | 11 | 0 | 0 | 105 |
| A05053HM | 0 | 0 | 4 | 0 | 0 | 23 |
| A05054H | 0 | 0 | 8 | 0 | 0 | 37 |
| A05055H | 0 | 0 | 3 | 0 | 0 | 34 |
| A05056H | 0 | 0 | 10 | 0 | 0 | 75 |
| A05057H | 0 | 0 | 10 | 0 | 0 | 40 |
| A05058H | 0 | 0 | 2 | 0 | 0 | 33 |
| A05059H | 0 | 0 | 4 | 0 | 0 | 12 |
| A05060H | 0 | 0 | 4 | 0 | 0 | 9 |
| A05061H | 0 | 0 | 8 | 0 | 0 | 25 |
| A05062H | 0 | 0 | 2 | 0 | 0 | 12 |
| A05063H | 0 | 0 | 5 | 0 | 0 | 7 |
| A05064H | 0 | 0 | 4 | 0 | 0 | 38 |
| A05065H | 0 | 0 | 0 | 0 | 0 | 6 |
| A05066H | 0 | 0 | 5 | 0 | 0 | 40 |
| A05067H | 0 | 0 | 8 | 0 | 0 | 83 |
| A05068H | 0 | 0 | 0 | 0 | 0 | 7 |
| A05069H | 0 | 0 | 4 | 0 | 0 | 23 |
| A05070H | 0 | 0 | 0 | 0 | 0 | 40 |
| A05071H | 0 | 0 | 0 | 0 | 0 | 14 |
| A05072H | 0 | 0 | 5 | 0 | 0 | 46 |
| A05073H | 0 | 0 | 3 | 0 | 0 | 16 |
| A05074H | 0 | 0 | 3 | 0 | 0 | 20 |
| A05075H | 0 | 0 | 9 | 0 | 0 | 42 |
| A05076H | 0 | 0 | 2 | 0 | 0 | 39 |
| A05077H | 0 | 0 | 5 | 0 | 0 | 28 |
| A05078H | 0 | 0 | 2 | 0 | 0 | 43 |
| A05079H | 0 | 0 | 4 | 0 | 0 | 16 |
| A05080H | 0 | 0 | 2 | 0 | 0 | 3 |
| A05081H | 0 | 0 | 0 | 0 | 0 | 6 |
| A05082H | 0 | 0 | 2 | 0 | 0 | 10 |
| A05083H | 0 | 0 | 3 | 0 | 0 | 21 |
| A05084H | 0 | 0 | 2 | 0 | 0 | 10 |
| A05085H | 0 | 0 | 3 | 0 | 0 | 36 |
| A05086H | 0 | 0 | 4 | 0 | 0 | 31 |
| A05087H | 0 | 0 | 1 | 0 | 0 | 17 |
| A05088H | 0 | 0 | 7 | 0 | 0 | 21 |
| A05089H | 0 | 0 | 4 | 0 | 0 | 17 |
| A05090H | 0 | 0 | 2 | 0 | 0 | 16 |
| A05091H | 0 | 0 | 6 | 0 | 0 | 57 |
| A05092H | 0 | 0 | 5 | 0 | 0 | 34 |
| A05093H | 0 | 0 | 0 | 0 | 0 | 11 |
| A05094H | 0 | 0 | 2 | 0 | 0 | 13 |
| A05095H | 0 | 0 | 1 | 0 | 0 | 16 |
| A05096H | 0 | 0 | 4 | 0 | 0 | 21 |
| A05097H | 0 | 0 | 1 | 0 | 0 | 18 |
| A05154H | 0 | 0 | 2 | 0 | 0 | 25 |
| A05155H | 0 | 0 | 5 | 0 | 0 | 19 |
| A05156H | 0 | 0 | 3 | 0 | 0 | 37 |
| A05157H | 0 | 0 | 2 | 0 | 0 | 32 |

TABLE 9
Number of genes, besides the target gene, that show a sequence
complementarity with the respective CD73 intron-binding
antisense oligonucleotide allowing 0, 1 or 2 mismatches.

| | |
|---|---|
| RefSeq (Gene Ids) | ENSEMBL |

| ASO | 0 mm | 1 mm | 2 mm | 0 mm | 1 mm | 2 mm |
|---|---|---|---|---|---|---|
| A05098HI | 0 | 0 | 3 | 0 | 0 | 87 |
| A05099HI | 0 | 0 | 22 | 0 | 0 | 41 |
| A05100HI | 0 | 0 | 1 | 0 | 0 | 24 |
| A05101HI | 0 | 0 | 3 | 0 | 0 | 43 |
| A05102HI | 0 | 0 | 13 | 0 | 0 | 92 |
| A05103HI | 0 | 0 | 1 | 0 | 0 | 21 |
| A05104HI | 0 | 0 | 8 | 0 | 0 | 47 |
| A05105HI | 0 | 0 | 13 | 0 | 0 | 117 |
| A05106HI | 0 | 0 | 6 | 0 | 0 | 35 |
| A05107HI | 0 | 0 | 7 | 0 | 0 | 32 |
| A05108HI | 0 | 0 | 5 | 0 | 0 | 32 |
| A05109HI | 0 | 0 | 3 | 0 | 0 | 24 |
| A05110HI | 0 | 0 | 2 | 0 | 0 | 8 |
| A05111HI | 0 | 0 | 3 | 0 | 0 | 18 |
| A05112HI | 0 | 0 | 1 | 0 | 0 | 16 |
| A05113HI | 0 | 0 | 4 | 0 | 0 | 41 |
| A05114HI | 0 | 0 | 4 | 0 | 0 | 42 |
| A05115HI | 0 | 0 | 3 | 0 | 0 | 27 |
| A05116HI | 0 | 0 | 5 | 0 | 0 | 50 |
| A05117HI | 0 | 0 | 1 | 0 | 0 | 27 |
| A05118HI | 0 | 0 | 3 | 0 | 0 | 25 |
| A05119HI | 0 | 0 | 0 | 0 | 0 | 8 |
| A05120HI | 0 | 0 | 0 | 0 | 0 | 8 |
| A05121HI | 0 | 0 | 4 | 0 | 0 | 25 |
| A05122HI | 0 | 0 | 8 | 0 | 0 | 44 |
| A05123HI | 0 | 0 | 5 | 0 | 0 | 45 |
| A05124HI | 0 | 0 | 1 | 0 | 0 | 26 |
| A05125HI | 0 | 0 | 4 | 0 | 0 | 50 |
| A05126HI | 0 | 0 | 2 | 0 | 0 | 29 |
| A05127HI | 0 | 0 | 1 | 0 | 0 | 9 |
| A05128HI | 0 | 0 | 4 | 0 | 0 | 6 |
| A05129HI | 0 | 0 | 8 | 0 | 0 | 38 |
| A05130HI | 0 | 0 | 0 | 0 | 0 | 6 |
| A05131HI | 0 | 0 | 6 | 0 | 0 | 20 |
| A05132HI | 0 | 0 | 3 | 0 | 0 | 23 |
| A05133HI | 0 | 0 | 1 | 0 | 0 | 6 |
| A05134HI | 0 | 0 | 2 | 0 | 0 | 11 |
| A05135HI | 0 | 0 | 7 | 0 | 0 | 40 |
| A05136HI | 0 | 0 | 4 | 0 | 0 | 10 |
| A05137HI | 0 | 0 | 5 | 0 | 0 | 33 |
| A05138HI | 0 | 0 | 1 | 0 | 0 | 18 |
| A05139HI | 0 | 0 | 3 | 0 | 0 | 43 |
| A05140HI | 0 | 0 | 2 | 0 | 0 | 17 |
| A05141HI | 0 | 0 | 1 | 0 | 0 | 39 |
| A05142HI | 0 | 0 | 1 | 0 | 0 | 25 |
| A05143HI | 0 | 0 | 2 | 0 | 0 | 23 |
| A05144HI | 0 | 0 | 1 | 0 | 0 | 22 |
| A05145HI | 0 | 0 | 6 | 0 | 0 | 20 |
| A05146HI | 0 | 0 | 9 | 0 | 0 | 30 |
| A05147HI | 0 | 0 | 1 | 0 | 0 | 61 |
| A05148HI | 0 | 0 | 8 | 0 | 0 | 37 |
| A05149HI | 0 | 0 | 1 | 0 | 0 | 21 |
| A05150HI | 0 | 0 | 9 | 0 | 0 | 89 |
| A05151HI | 0 | 0 | 2 | 0 | 0 | 19 |
| A05152HI | 0 | 0 | 4 | 0 | 0 | 40 |
| A05153HI | 0 | 0 | 6 | 0 | 0 | 37 |

### Example 5: In Vivo Assessment of Liver Toxicity of Selected Antisense Oligonucleotides

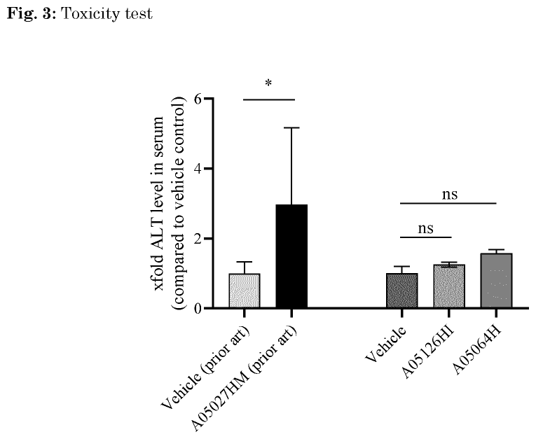

In order to determine the liver toxic capacity of the antisense oligonucleotides A05027HM (control (SEQ ID NO.116), see e.g., WO2018/065627), A05126HI (SEQ ID NO.83) and A05064H (SEQ ID NO.21) of the present invention mice were treated with repeated injections (20 mg/kg) of the respective antisense oligonucleotide. The serum levels of Alanine transaminase were determined at different time points. As shown in FIG. 3 and Table 10, treatment of mice with A05027HM (control of prior art, SEQ ID NO.116) led to a significant increase of ALT on day 5 as compared to vehicle control mice. In contrast, treatment with none of the two tested ASOs of the present invention led to a significant increase of ALT as compared to vehicle control animals.

TABLE 10
Liver toxicity of selected ASOs.

| | Mean x-fold ALT | |
|---|---|---|
| | level in serum | p value |
| | (compared to | (compared to |
| | vehicle control) | vehicle control) |
| A05027HM (SEQ ID NO. 116 | 2.97 | 0.02 |
| of prior art) | | |
| A05126HI (SEQ ID NO. 83) | 1.26 | 0.9 |
| A05064H (SEQ ID NO. 21) | 1.58 | 0.6 |

1. An oligonucleotide comprising 12 to 20 nucleotides, wherein at least one of the nucleotides is modified, and the oligonucleotide hybridizes with a nucleic acid sequence of an ectoenzyme (CD73) pre-mRNA of SEQ ID NO.2 in a hybridizing active region selected from the group consisting of position 25201 to 25600, position 4401 to 4800, position 44401 to 44800, position 16801 to 17200, position 21201 to 21600, position 801 to 1200, position 19201 to 19600, position 20801 to 21200, position 1 to 400, position 44001 to 44400, position 44801 to 45200, position 401 to 800, position 44401 to 44800, position 2401 to 2800, position 41601 to 42000, position 43601 to 44000, position 45201 to 45701, position 38001 to 38400, position 2801 to 3200, position 40401 to 40800, position 1601 to 2000, position 22001 to 22400, position 42401 to 42800, position 22401 to 22800, position 42801 to 43200, position 37601 to 3800, position 39201 to 39600, position 19201 to 19600, position 35201 to 35600, position 40001 to 40400, position 42001 to 42400, position 41201 to 41600, position 2001 to 2400, position 20001 to 20400, position 17201 to 17600, position 38801 to 39200, position 37201 to 37600, position 39601 to 40000, position 24801 to 25200, position 38401 to 38800, position 43201 to 43600, position 36801 to 37200, position 4001 to 4400, position 35601 to 36000, position 36001 to 36400, position 18401 to 18800, position 40801 to 41200, 4401 to 4800, position 16801 to 17200, position 25201 to 25600 and a combination thereof.

2. The oligonucleotide of claim 1, wherein the modified nucleotide is selected from the group consisting of a bridged nucleic acid such as LNA, cET, ENA, 2′Fluoro modified nucleotide, 2′O-Methyl modified nucleotide and combinations thereof.

3. The oligonucleotide of claim 1 or 2, wherein the oligonucleotide reduces the amount of CD73 mRNA or the level of CD73 protein expression for at least 50% compared to an untreated control.

4. The oligonucleotide of any one of claims 1 to 3, wherein the oligonucleotide is selected from the group consisting of SEQ ID NO.83, SEQ ID NO.71, SEQ ID NO.47, SEQ ID NO.21, SEQ ID NO.22, SEQ ID NO.112, SEQ ID NO.72, SEQ ID NO.14, SEQ ID NO.46, SEQ ID NO.48, SEQ ID NO.49, SEQ ID NO.24, SEQ ID NO.25, SEQ ID NO.113, SEQ ID NO.64, SEQ ID NO.55, SEQ ID NO.76, SEQ ID NO.23, SEQ ID NO.59, SEQ ID NO.78, SEQ ID NO.3, SEQ ID NO.4, SEQ ID NO.5, SEQ ID NO.6, SEQ ID NO.16, SEQ ID NO.17, SEQ ID NO.18, SEQ ID NO.19, SEQ ID NO.20, SEQ ID NO.11, SEQ ID NO.50, SEQ ID NO.51, SEQ ID NO.52, SEQ ID NO.53, SEQ ID NO.54, SEQ ID NO.63, SEQ ID NO.14, SEQ ID NO.46, SEQ ID NO.48, SEQ ID NO.49, SEQ ID NO.57, SEQ ID NO.66, SEQ ID NO.67, SEQ ID NO.108, SEQ ID NO.109, SEQ ID NO.9, SEQ ID NO.10, SEQ ID NO.11, SEQ ID NO.38, SEQ ID NO.39, SEQ ID NO.12, SEQ ID NO.40, SEQ ID NO.41, SEQ ID NO.42, SEQ ID NO.43, SEQ ID NO.106, SEQ ID NO.15, SEQ ID NO.60, SEQ ID NO.90, SEQ ID NO.91, SEQ ID NO.68, SEQ ID NO.69, SEQ ID NO.8, SEQ ID NO.36, SEQ ID NO.37, SEQ ID NO.107, SEQ ID NO.79, SEQ ID NO.101, SEQ ID NO.102, SEQ ID NO.80, SEQ ID NO.81, SEQ ID NO.56, SEQ ID NO.62, SEQ ID NO.103, SEQ ID NO.88, SEQ ID NO.89, SEQ ID NO.7, SEQ ID NO.34, SEQ ID NO.94, SEQ ID NO.95, SEQ ID NO.55, SEQ ID NO.76, SEQ ID NO.27, SEQ ID NO.28, SEQ ID NO.29, SEQ ID NO.30, SEQ ID NO.31, SEQ ID NO.61, SEQ ID NO.97, SEQ ID NO.114, SEQ ID NO.99, SEQ ID NO.100, SEQ ID NO.65, SEQ ID NO.58, SEQ ID NO.77, SEQ ID NO.110, SEQ ID NO.73, SEQ ID NO.74, SEQ ID NO.93, SEQ ID NO.33, SEQ ID NO.96, SEQ ID NO.82, SEQ ID NO.92, SEQ ID NO.104, SEQ ID NO.105, SEQ ID NO.86, SEQ ID NO.87, SEQ ID NO.70, SEQ ID NO.84, SEQ ID NO.85, SEQ ID NO.75, SEQ ID NO.98 and combinations thereof.

5. The oligonucleotide of any one of claims 1 to 4, wherein the oligonucleotide is selected from the group consisting of
(A05126HI, SEQ ID NO. 83)
+G*+A*+C*T*C*T*A*G*G*A*T*T*T*A*A*+C*+T*+T,
(A05114HI, SEQ ID NO. 71)
+A*+G*+A*A*G*T*G*A*A*T*T*G*C*A*T*+A*+G*+C,
(A05090H, SEQ ID NO. 47)
+G*+A*+T*T*T*G*T*C*A*T*C*C*A*T*G*+A*+G*+C,
(A05064H, SEQ ID NO. 21)
+G*+G*+A*A*A*T*T*T*G*G*C*C*T*C*T*+T*+T*+G,
(A05065H, SEQ ID NO. 22)
+A*+A*+G*T*G*T*A*T*C*C*A*A*C*G*A*+T*+T*+C,
(A05155H, SEQ ID NO. 112)
+G*+T*+G*T*A*T*C*C*A*A*C*G*A*T*+T*+C*+C,
(A05115HI, SEQ ID NO. 72)
+C*+A*+G*A*G*G*T*A*A*G*C*T*G*G*T*+T*+C*+A,
(A05057H, SEQ ID NO. 14)
+C*+T*+C*G*T*G*T*C*C*T*T*T*G*A*+C*+T*+G,
(A05089H, SEQ ID NO. 46)
+G*+G*+A*T*C*T*G*T*C*A*G*C*A*T*T*+A*+G*+C,
(A05091H, SEQ ID NO. 48)
+A*+C*+T*C*A*T*C*A*A*A*G*G*C*A*C*+A*+T*+G,
(A05092H, SEQ ID NO. 49)
+A*+T*+T*A*T*C*T*A*C*T*A*C*A*G*C*+T*+T*+G,
(A05067H, SEQ ID NO. 24)
+T*+T*+T*G*T*T*C*A*C*A*T*T*T*A*G*+A*+G*+T,
(A05068H, SEQ ID NO. 25)
+C*+T*+T*T*C*T*G*A*G*C*G*A*T*G*A*+G*+T*+T,
(A05156H, SEQ ID NO. 113)
+G*+C*+G*A*T*G*A*G*T*T*T*A*T*C*+C*+A*+T,
(A05107HI, SEQ ID NO. 64)
+G*+A*+T*C*T*G*G*T*G*T*C*C*A*T*T*+C*+T*+T,
(A05098HI, SEQ ID NO. 55)
+C*+G*+A*G*A*G*T*A*T*G*C*T*A*+C*+C*+A,
(A05119H, SEQ ID NO. 76)
+A*+T*+G*T*C*G*A*G*A*G*T*A*T*G*C*+T*+A*+C,
(A05066H, SEQ ID NO. 23)
+A*+T*+C*T*A*C*T*T*C*A*G*G*T*T*G*+T*+A*+A,
(A05102H, SQE ID NO.59)
+T*+G*+G*T*T*G*C*A*A*A*C*T*G*T*+G*+A*+G,
(A05121HI, SEQ ID NO. 78)
+G*+G*+A*G*G*C*T*T*C*A*G*A*T*A*T*+T*+G*+T,
(A05046H, SEQ ID NO. 3)
+G*+C*+G*A*G*T*G*C*C*G*G*C*G*+A*+G*+T,
(A05047H, SEQ ID NO. 4)
+A*+G*+C*T*G*T*G*G*C*G*C*G*T*G*+A*+A*+C,
(A05048H, SEQ ID NO. 5)
+G*+C*+T*G*G*C*G*T*T*G*A*C*G*C*+A*+C*+T,
(A05049H, SEQ ID NO. 6)
+C*+C*+T*G*G*T*A*C*T*G*G*T*C*G*+C*+C*+G,
(A05059H, SEQ ID NO. 16)
+G*+G*+A*C*A*C*A*T*A*G*C*T*G*T*G*+G*+C*+G,
(A05060H, SEQ ID NO. 17)
+C*+T*+G*T*G*C*A*C*G*T*C*G*T*T*G*+G*+T*+G,
(A05061H, SEQ ID NO. 18)
+T*+G*+G*A*G*T*C*C*T*C*G*C*T*G*G*+T*+C*+T,
(A05062H, SEQ ID NO. 19)
+T*+T*+G*A*C*G*C*A*C*T*T*G*C*T*G*+G*+A*+G,
(A05063H, SEQ ID NO. 20)
+A*+T*+G*G*C*A*T*C*G*T*A*G*C*G*C*+A*+G*+G,
(A05054H, SEQ ID NO. 11)
+C*+T*+T*A*T*A*T*A*C*C*T*C*G*T*+C*+C*+A,
(A05093H, SEQ ID NO. 50)
+A*+T*+A*A*C*A*G*C*T*A*A*T*G*C*C*+G*+T*+G,
(A05094H, SEQ ID NO. 51)
+G*+C*+T*T*A*T*G*T*T*A*G*A*A*G*G*+T*+T*+C,
(A05095H, SEQ ID NO. 52)
+T*+C*+G*A*G*A*A*C*T*C*T*G*G*A*C*+A*+T*+T,
(A05096H, SEQ ID NO. 53)
+T*+G*+G*A*G*G*C*A*G*A*G*C*G*A*C*+T*+T*+A,
(A05097H, SEQ ID NO. 54)
+G*+G*+C*A*T*A*G*G*T*C*A*T*T*T*C*+A*+T*+C,
(A05106HI, SEQ ID NO. 63)
+C*+A*+C*A*C*T*C*T*G*C*C*A*T*C*C*+G*+C*+T,
(A05057H, SEQ ID NO. 14)
+C*+T*+C*G*T*G*T*C*C*T*T*T*G*A*+C*+T*+G,
(A05089H, SEQ ID NO. 46)
+G*+G*+A*T*C*T*G*T*C*A*G*C*A*T*T*+A*+G*+C,
(A05091H, SEQ ID NO. 48)
+A*+C*+T*C*A*T*C*A*A*A*G*G*C*A*C*+A*+T*+G,
(A05092H, SEQ ID NO. 49)
+A*+T*+T*A*T*C*T*A*C*T*A*C*A*G*C*+T*+T*+G,
(A05100HI, SEQ ID NO. 47)
+G*+A*+A*T*C*A*A*T*A*T*G*C*G*G*+T*+G*+A,
(A05109HI, SEQ ID NO. 66)
+G*+C*+C*A*T*C*T*A*A*C*C*T*T*G*C*+C*+A*+T,
(A05110HI, SEQ ID NO. 67)
+A*+T*+C*A*A*T*A*T*G*C*G*G*T*G*A*+G*+T*+G,
(A05151HI, SEQ ID NO. 108)
+A*+T*+C*A*A*T*A*T*G*C*G*G*T*G*+A*+G*+T,
(A05152HI, SEQ ID NO. 109)
+C*+T*+A*G*G*A*A*T*C*A*A*T*A*T*+G*+C*+G,
(A05052H, SEQ ID NO. 9)
+T*+C*+A*T*A*C*A*C*C*A*C*A*T*G*+G*+A*+T,
(A05053HM, SEQ ID NO. 10)
+G*+G*+C*A*C*T*C*G*A*C*A*C*T*T*+G*+G*+T,
(A05054H, SEQ ID NO. 11)
+C*+T*+T*A*T*A*T*A*C*C*T*C*G*T*+C*+C*+A,
(A05081H, SEQ ID NO. 38)
+C*+G*+A*C*A*C*T*T*G*G*T*G*C*A*A*+A*+G*+A,
(A05082H, SEQ ID NO. 39)
+A*+T*+A*C*C*T*C*G*T*C*C*A*T*T*T*+T*+G*+A,
(A05055H, SEQ ID NO. 12)
+G*+T*+A*G*A*A*A*C*C*A*C*G*T*T*+G*+A*+T,
(A05083H, SEQ ID NO. 40)
+C*+G*+A*C*C*T*T*C*A*A*C*T*G*C*T*+G*+G*+A,
(A05084H, SEQ ID NO. 41)
+A*+A*+C*T*T*G*A*T*C*C*G*A*C*C*T*+T*+C*+A,
(A05085H, SEQ ID NO. 42)
+C*+C*+T*G*T*G*G*A*A*A*A*C*T*T*G*+A*+T*+C,
(A05086H, SEQ ID NO. 43)
+G*+G*+T*C*C*T*A*A*A*A*G*G*C*A*G*+A*+T*+T,
(A05149HI, SEQ ID NO. 106)
+G*+G*+T*A*G*T*C*C*T*T*T*G*T*A*A*+T*+T*+A,
(A05058H, SEQ ID NO. 15)
+T*+A*+G*A*A*C*C*G*A*G*G*C*T*A*+T*+T*+A,
(A05103HI, SEQ ID NO. 60)
+G*+T*+A*G*T*C*C*G*A*C*A*T*A*G*+G*+A*+G,
(A05133HI, SEQ ID NO. 90)
+G*+T*+A*G*T*C*C*G*A*C*A*T*A*G*G*+A*+G*+A,
(A05134HI, SEQ ID NO. 91)
+T*+T*+G*G*C*A*T*G*A*G*C*A*T*G*A*+T*+T*+G,
(A05111HI, SEQ ID NO. 68)
+G*+G*+C*T*C*C*T*T*T*G*A*A*C*T*A*+G*+G*+T,
(A05112HI, SEQ ID NO. 69)
+C*+T*+A*G*A*A*A*G*T*G*T*A*C*A*C*+C*+T*+C,
(A05051H, SEQ ID NO. 8)
+C*+A*+C*G*C*T*A*T*G*C*T*C*A*A*+A*+G*+G,
(A05079H, SEQ ID NO. 36)
+G*+T*+G*G*A*A*C*C*T*T*T*T*A*A*C*+T*+G*+G,
(A05080H, SEQ ID NO. 37)
+A*+G*+T*G*G*A*C*T*G*G*C*C*G*T*A*+G*+C*+G,
(A05150HI, SEQ ID NO. 107)
+A*+C*+C*A*A*T*G*C*T*T*A*A*G*A*+G*+A*+C,
(A05122HI, SEQ ID NO. 79)
+A*+A*+G*G*T*G*G*A*A*C*C*A*G*A*T*+T*+C*+A,
(A05144HI, SEQ ID NO. 101)
+G*+T*+C*A*G*T*G*G*T*C*T*G*T*A*T*+G*+C*+A,
(A05145HI, SEQ ID NO. 102)
+T*+T*+G*T*A*A*G*C*A*T*G*C*T*G*G*+T*+C*+T,
(A05123HI, SEQ ID NO. 80)
+T*+G*+G*A*A*C*T*T*T*G*A*G*C*A*T*+G*+A*+T,
(A05124HI, SEQ ID NO. 81)
+C*+T*+T*A*A*G*T*G*A*A*G*G*C*C*A*+A*+C*+T,
(A05099HI, SEQ ID NO. 56)
+T*+G*+C*G*G*C*C*G*A*G*C*C*A*+T*+T*+G,
(A05105HI, SEQ ID NO. 62)
+G*+A*+G*C*C*A*T*T*G*G*T*A*T*T*+T*+A*+A,
(A05146HI, SEQ ID NO. 103)
+G*+A*+G*C*C*A*T*T*G*G*T*A*T*T*T*+A*+A*+T,
(A05131HI, SEQ ID NO. 88)
+G*+G*+T*A*C*T*G*A*G*T*A*T*G*A*A*+G*+C*+T,
(A05132HI, SEQ ID NO. 89)
+G*+T*+A*G*C*A*G*A*G*T*T*T*G*T*G*+C*+A*+T,
(A05050H, SEQ ID NO. 7)
+C*+C*+G*T*G*T*G*T*C*T*C*A*G*G*+T*+T*+G,
(A05077H, SEQ ID NO. 34)
+G*+G*+A*T*A*C*C*A*C*C*T*C*C*A*T*+T*+T*+A,
(A05137HI, SEQ ID NO. 94)
+C*+C*+A*G*T*C*C*A*T*G*T*C*A*A*A*+C*+T*+C,
(A05138HI, SEQ ID NO. 95)
+G*+A*+T*T*T*A*C*A*C*T*A*G*T*T*A*+C*+T*+C,
(A05098HI, SEQ ID NO. 55)
+C*+G*+A*G*A*G*T*A*T*G*C*T*A*+C*+C*+A,
(A05119HI, SEQ ID NO. 76)
+A*+T*+G*T*C*G*A*G*A*G*T*A*T*G*C*+T*+A*+C,
(A05070H, SEQ ID NO. 27)
+C*+T*+A*C*A*G*G*A*A*C*C*T*T*C*C*+G*+C*+C,
(A05071H, SEQ ID NO. 28)
+G*+C*+A*T*A*G*G*C*C*T*G*G*A*C*T*+A*+C*+A,
(A05072H, SEQ ID NO. 29)
+T*+T*+C*A*G*A*T*A*G*C*C*T*A*G*G*+T*+A*+T,
(A05073H, SEQ ID NO. 30)
+A*+A*+C*T*C*G*A*T*C*T*T*C*A*G*A*+T*+A*+G,
(A05074H, SEQ ID NO. 31)
+C*+T*+C*T*T*T*C*A*T*C*A*A*A*C*T*+C*+G*+A,
(A05104HI, SEQ ID NO. 61)
+T*+T*+A*G*A*T*C*T*G*C*T*A*G*C*+T*+T*+G,
(A05140HI, SEQ ID NO. 97)
+C*+T*+T*A*G*A*T*C*T*G*C*T*A*G*C*+T*+T*+G,
(A05157H, SEQ ID NO. 114)
+A*+C*+C*T*T*A*T*A*T*A*C*C*T*C*+G*+T*+C,
(A05142HI, SEQ ID NO. 99)
+A*+T*+G*C*A*G*T*G*C*T*T*T*G*C*T*+A*+G*+A,
(A05143HI, SEQ ID NO. 100)
+C*+A*+C*A*A*G*G*C*A*T*A*G*A*G*C*+T*+A*+T,
(A05108HI, SEQ ID NO. 65)
+T*+A*+G*C*T*G*T*G*G*A*A*T*A*C*C*+A*+A*+T,
(A05101HI, SEQ ID NO. 58)
+G*+T*+A*A*C*A*A*A*C*G*A*T*A*G*+C*+C*+T,
(A05120HI, SEQ ID NO. 77)
+A*+G*+A*T*C*C*A*G*A*C*G*T*T*C*T*+T*+A*+C,
(A05153HI, SEQ ID NO. 110)
+T*+G*+G*T*A*A*C*A*A*A*C*G*A*T*+A*+G*+C,
(A05116HI, SEQ ID NO. 73)
+A*+T*+G*C*C*A*A*G*C*T*G*T*G*A*T*+T*+T*+A,
(A05117HI, SEQ ID NO. 74)
+C*+T*+G*T*T*T*A*G*C*A*C*T*G*G*C*+T*+A*+T,
(A05136HI, SEQ ID NO. 93)
+A*+A*+T*G*G*T*C*T*C*T*C*G*G*T*T*+G*+T*+A,
(A05076H, SEQ ID NO. 33)
+A*+G*+G*A*G*C*C*A*T*C*C*A*G*A*T*+A*+G*+A,
(A05139HI, SEQ ID NO. 96)
+T*+T*+T*A*A*T*C*C*A*G*T*G*G*T*A*+T*+G*+T,
(A05125HI, SEQ ID NO. 82)
+A*+A*+T*C*C*A*C*G*A*G*C*T*T*T*G*+G*+A*+A,
(A05135HI, SEQ ID NO. 92)
+G*+A*+T*A*A*G*C*A*C*T*G*C*C*A*A*+C*+A*+G,
(A05147HI, SEQ ID NO. 104)
+G*+A*+T*A*A*A*T*G*C*T*A*A*T*T*G*+C*+C*+T,
(A05148HI, SEQ ID NO. 105)
+T*+T*+G*A*A*C*C*A*C*T*C*C*A*G*A*+A*+C*+A,
(A05129HI, SEQ ID NO. 86)
+C*+A*+T*C*T*G*G*C*T*A*C*T*G*A*G*+A*+G*+G,
(A05130HI, SEQ ID NO. 87)
+A*+T*+T*A*G*T*G*G*T*G*G*C*G*G*T*+A*+G*+G,
(A05113HI, SEQ ID NO. 70)
+C*+C*+T*A*C*A*A*T*A*A*A*G*C*T*G*+G*+A*+T,
(A05127HI, SEQ ID NO. 84)
+C*+C*+G*C*A*A*T*A*G*A*C*T*C*A*G*+A*+C*+A,
(A05128HI, SEQ ID NO. 85)
+A*+C*+G*C*T*C*A*T*C*T*T*G*C*C*G*+C*+C*+G,
(A05118HI, SEQ ID NO. 75)
+G*+T*+T*A*C*A*G*C*C*T*G*G*T*A*A*+A*+G*+G,
(A05141HI, SEQ ID NO. 98)
+T*+A*+T*T*A*G*A*A*A*C*T*A*G*A*C*+C*+T*+C
and combinations thereof, wherein + indicates an LNA nucleotide and * indicates a phosphorothioate (PTO) linkage between the nucleotides.

6. The oligonucleotide of any one of claims 1 to 5, wherein the oligonucleotide reduces the amount of CD73 mRNA or the level of CD73 protein expression at a nanomolar concentration.

7. A pharmaceutical composition comprising an oligonucleotide of any one of claims 1 to 6 and a pharmaceutically acceptable carrier, excipient and/or dilutant.

8. The pharmaceutical composition of claim 7, further comprising an antitumor active agent such as a chemotherapeutic (e.g., platinum, gemcitabine), an immune stimulating agent, disease specific agent or an agent that reverses tumor- or infection-mediated immunosuppression, another oligonucleotide, an antibody, a carbohydrate-modified antibody, a peptide-based therapeutic, a protein-based therapeutic, a lipid, a therapeutic vaccine, a HERA fusion protein, a ligand trap, a Fab fragment, a nanobody, a BiTe, a DARPin, a small molecule or a combination thereof.

9. The pharmaceutical composition of claim 8, wherein the antitumor active agent, the disease specific agent, the other oligonucleotide, the antibody, the carbohydrate-modified antibody, the peptide-based therapeutic, the protein-based therapeutic, the lipid, the therapeutic vaccine, the HERA fusion protein, the ligand trap, the Fab fragment, the nanobody, the BiTe, the DARPin and/or the small molecule inhibits expression or activity of an immune suppressive factor selected from the group consisting of IDO1, IDO2, CTLA-4, PD-1, PD-L1, LAG-3, VISTA, A2AR, CD39, CD73, STAT3, TDO2, TIM-3, TIGIT, TGF-beta, BTLA, MICA, NKG2A, KIR, CD160, MTDH, Xbp1, Chop and a combination thereof, or stimulates expression or activity of an immune stimulatory factor selected from the group consisting of 4-1BB, Ox40, KIR, GITR, CD27, 2B4 and a combination thereof.

10. The pharmaceutical composition of claim 8 or 9, wherein the antitumor active agent, the disease specific agent, the other oligonucleotide, the antibody, the carbohydrate-modified antibody, the peptide-based therapeutic, the protein-based therapeutic, the lipid, the therapeutic vaccine, the HERA fusion protein, the ligand trap, the Fab fragment, the nanobody, the BiTe, the DARPin and/or the small molecule inhibits expression or activity of a factor involved in cancer progression and/or metastasis selected from the group consisting of SND1, MTDH, HER-2, BRAF, KRAS, VEGF, EGFR1, EGFR2, BCR/ABL, ABL, MET, ALK, JAK2, BTK, miR-223, CCL18, CCL20, Lcn2, CCL5/CCR9, DDR2, PHD2, IL6, SDF-1/CXCL12 and a combination thereof.

11. The oligonucleotide of any one of claims 1 to 6 or the pharmaceutical composition of any one of claims 7 to 10 for use in a method of preventing and/or treating a disorder, where a CD73 imbalance is involved.

12. The oligonucleotide or the pharmaceutical composition for use according to claim 11, wherein the disorder is an autoimmune disorder, an immune disorder, a psychiatric disorder and/or cancer.

13. The oligonucleotide or the pharmaceutical composition for use according to claim 12, wherein the cancer is breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomater tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythemia vera, adenocarcinoma, anaplastic astrocytoma, glioblastoma multiforme, leukemia, or epidermoid carcinoma.

14. The oligonucleotide or the pharmaceutical composition for use according to any one of claims 11 to 13, wherein the oligonucleotide or the pharmaceutical composition is administered locally or systemically.
